(19) United States
(12) Patent Application Publication
(10) Pub. No.: US 2014/0018641 A1
(43) Pub. Date: Jan. 16, 2014

(54) MOISTURE METER AND BODY MOISTURE METER

(71) Applicant: TERUMO KABUSHIKI KAISHA, Shibuya-ku (JP)

(72) Inventors: Keisuke YOSHINO, Kanagawa (JP); Miyuki KOYAMA, Kanagawa (JP); Kiyoyuki NARIMATSU, Kanagawa (JP); Takashi MORITA, Kanagawa (JP)

(73) Assignee: TERUMO KABUSHIKI KAISHA, Shibuya-ku (JP)

(21) Appl. No.: 14/026,028

(22) Filed: Sep. 13, 2013

Related U.S. Application Data
(63) Continuation of application No. PCT/JP2012/001794, filed on Mar. 14, 2012.

(30) Foreign Application Priority Data
Mar. 15, 2011 (JP) 2011-056586

(51) Int. Cl. A61B 5/00; A61B 5/01

(52) U.S. Cl. CPC A61B 5/4875; A61B 5/443; A61B 5/01; A61B 5/6813; A61B 5/742; A61B 5/746
USPC 600/301; 600/306

(57) ABSTRACT

To provide a moisture meter which can detect a heat-illness risk-index earlier and which is effective to assist a subject carry out a proper moisture adjustment includes: a moisture measurement unit held by an armpit of a subject and which measures the amount of moisture of the subject through contact with a skin surface of the armpit, a sensor unit which measures the temperature and humidity of the environment of the subject, and a processing unit which obtains the amount of moisture of the subject from the moisture measurement unit, which sets a Wet-Bulb Globe temperature (WBGT) value from the relationship between the temperature and humidity from the sensor unit and which obtains the heat-illness risk-index by referring to a relation table between the amount of moisture of the subject and the Wet-Bulb Globe temperature value.

### CROSS REFERENCES TO RELATED APPLICATIONS

This application is a continuation of International Application No. PCT/JP2012/001794 filed on Mar. 14, 2012, and claims priority to Japanese Application No. 2011-056586 filed on Mar. 15, 2011, the entire content of both of which is incorporated herein by reference.

### TECHNICAL FIELD

The present invention generally relates to a moisture meter and a body moisture meter, which measure moisture content of a living body by being sandwiched by an armpit of a subject.

### BACKGROUND DISCUSSION

It is important to measure moisture content of a living body of a subject. Dehydration in a living body is a pathologic condition in which the moisture inside the living body decreases, and it is a symptom which is developed often in a daily life and is developed often, in particular, during exercise, in which much moisture is discharged from the inside of the body to the outside of the body caused by sudation and by increase of the body-temperature, and when the air-temperature is high. In particular, there are many instances in which the moisture holding ability of aged persons deteriorates, and dehydration amongst aged persons more easily results as compared with general healthy persons.

In general, in an aged person, muscle-stored water decreases, urine volume increases due to the lowering of the kidney function, it is more difficult to notice the mouth thirst caused by the slowdown feeling, the moisture required for the inside of the cell becomes less, etc. When this dehydration is left unattended, there sometimes happens a case in which the dehydration becomes a trigger and a serious symptom will make progress. In addition, a similar dehydration can be seen also for an infant. For an infant, the amount of moisture is a lot originally, but the infant is unable to resupply lost moisture by himself/herself and so situations sometimes occur in which the dehydration occurs because there is a delay before the infant's guardian notices the dehydrated condition.

Generally, it is said that a failure of body-temperature adjustment will occur when the moisture inside the living body is reduced by 2% or more of the body weight, wherein the failure of the body-temperature adjustment causes body-temperature increase, the body-temperature increase falls into a vicious cycle which causes further moisture decrease inside the living body and lastly, there occurs a situation of reaching a pathologic condition referred to as a heat illness. In the heat illness, there exists pathologic conditions such as heat cramp, heat exhaustion, heat stroke and the like, wherein in some cases, there may occur a situation in which organopathy of the whole body is caused. It is desirable to comprehend the dehydration accurately to prevent the risk of reaching the heat illness from occurring.

As an apparatus for realizing dehydration, there is known an apparatus in which the impedance of the human body is measured by using an apparatus whose handles are held by both hands and the amount of moisture is to be calculated therefrom. Examples are disclosed in Japanese Application Publication No. H11-318845, Japanese Patent No. 3977983 and Japanese Patent No. 3699640.

Another apparatus for realizing dehydration is an oral-cavity moisture meter or the like which measures the moisture content inside an oral cavity such as a tongue mucosa, a cheek mucosa, a palate or the like. Examples are disclosed in International Application Publication No. WO2004/028359, Japanese Application Publication No. 2001-170088 and Japanese Application Publication No. 2005-287547.

Further, known methods for measuring the amount of skin moisture include, starting from an in-vitro weighting method and a Karl Fischer method, an in-vivo ATR spectroscopic method and which moreover include a high-frequency impedance method and an electrical conductivity method which are easier-to-use in-vivo measuring methods.

However, the moisture meter, which measures impedance of a human body using an apparatus whose handles are held by both hands and which calculates the amount of moisture from the impedance of the human body, measures the impedance from the hand skin, so that this is easily affected by the skin humidity, the amount of arm muscle or the like, in which for an aged person or for a person having a handicap in his body, the apparatus is large-sized, the measurement must be carried out in a standing state and so on. The use performance of these apparatus is thus not very good.

In general, it is known that when the body-temperature varies, the bioelectrical-impedance value, that is, the amount of moisture varies. When the body-temperature increases, the bioelectrical-impedance value declines and when the body-temperature declines, the bioelectrical-impedance value increases. However, according to the know moisture meter, the amount of body moisture is calculated from the measured bioelectrical-impedance value without considering anything about the fact that bioelectrical-impedance value varies in this manner caused by the change of the body-temperature, so that it is not possible to determine an accurate amount of body moisture and therefore, it is not possible to detect the dehydration accurately. For example, in a case in which the amount of body moisture decreases and the body-temperature increases, the bioelectrical-impedance value increases caused by the decrease of the amount of body moisture, but the bioelectrical-impedance value declines caused by the body-temperature increase, so that even if the judgment is carried out from the amount of body moisture which is calculated from the measured bioelectrical-impedance value, there may occur a situation that the dehydrated state is not to be detected. For this reason, in case of carrying out the measurement by an impedance method, it is necessary to comprehend how much degree the body-temperature of the measured person is, but there has not been carried out a correction for the impedance value according to the measurement of the body-temperature or there has not been carried out an alarm or the like such as a description that an accurate amount of moisture cannot be judged because of developing a fever.

Also, the oral-cavity moisture meter which measures the moisture content inside an oral cavity such as a tongue mucosa, a cheek mucosa, a palate or the like must be attached with a newly exchangeable cover for every subject at the portion which is inserted directly to the inside of the oral cavity in order to prevent the transfer of infection between subjects. There is also a possibility of forgetting the fact that the cover should be attached by being exchanged and so the use performance is bad for aged persons or persons having a bodily handicap.

The dehydrated state judging apparatus described in the Japanese Patent No. 3977983 is an apparatus which judges the dehydration state based on the bioelectrical-impedance value in consideration of the body-temperature such as an apparatus which is provided with a body-temperature sensor for carrying out the body-temperature measurement through a thumb and in which based on this body-temperature, the measurement value of the bioelectrical impedance is corrected. Based on this corrected bioelectrical-impedance value, the judgment of the dehydrated state is carried out so that it is possible to judge the dehydrated state more accurately and it is possible for the subject to inspect his dehydration state accurately.

However, in this document, the body-temperature is measured at a thumb in which there is difficulty in the body-temperature measurement by the thumb and this is not a practical technique.

At the medical forefront, dehydration is judged by a number of methods. For example, a method expressing dehydration according to the blood collection data, the judgment thereof is carried out based on a high value of hematocrit, a high value of sodium, 25 mg/dL or more of urea nitrogen, 25 or more of urea nitrogen/creatinine ratio, 7 mg/dl or more of uric acid level, or the like. However, it is necessary for this method to collect blood and it is not possible to use this method at home or the like.

Other judgment methods include a dry state of the tongue or the inside of the oral cavity; a dry state of the armpit; lowering of willingness such as a phenomenon of “lacking in energy somehow”; blunting of consciousness such as a phenomenon of “being exhausted and reaction is dull”; and the like. But each of these alternatives requires the intuition and experience of a healthcare worker and is not a judgment that can be handled by anybody.

In addition, in case of the body moisture meter described in Japanese Application Publication No. H11-318845, the subject must grasp the handles with both hands, and so there is a problem that it is not possible for a third person (measurer) other than the subject to measure the amount of body water of the subject. More specifically, according to the structure of the body moisture meter which is on an assumption of the measurement region described in Japanese Application Publication No. H11-318845, there is such a problem that it is not possible for the third person (measurer) to measure the amount of body water of the subject who, for example, falls out of consciousness.

An example of another measurement region for which measurement can be carried out by a third person (measurer) other than the subject and which is suitable for the measurement of the amount of body water is the skin of an armpit. However, in case of a subject such as an aged person whose armpit is deep, it is not always easy to press the sensor unit of the body moisture meter accurately onto the armpit. Consequently, for the body moisture meter in which the armpit is made to be the measurement region, it is important to employ a structure in which the measurement is easy for the measurer regardless of (the physical feature of) the subject.

In addition, in case of the body moisture meter described in Japanese Application Publication No. H11-318845, the subject himself must grasp the handles with both hands, and so it is not possible for a third person (measurer) other than the subject to measure the amount of body water of the subject. More specifically, according to the structure of the body moisture meter described in Japanese Application Publication No. H11-318845, it is not possible for the third person (measurer) to measure the amount of body water of the subject who, for example, falls into a disturbance of consciousness (i.e., becomes unconscious).

On the other hand, skin of an armpit can be sued as the measurement region for which measurement can be carried out by a third person (measurer) other than the subject, and also, which is suitable for the measurement of the amount of body water. In case of measuring the amount of body water in the armpit, it is desirable to provide operability such as, for example, of a body-temperature meter, and also to be able to judge whether or not there is a tendency of dehydration by a simple and convenient configuration such as that of the body-temperature measurement by the body-temperature meter. In general, with regard to the body-temperature, the vicinity of 37° C. has been established as the boundary whether or not it is a normal body-temperature and it is possible for the user who measures the body-temperature to carry out a rough judgment intuitively whether or not fever is developed by making the vicinity of 37° C. as a target. However, with regard to the amount of body water, there is no widely established target, and even if it is possible to measure the amount of body water easily and to comprehend the significance of the numerical value, it is difficult to judge intuitively from the numerical value whether or not it indicates a dehydrated state or the degree of the dehydrated state.

### SUMMARY

A first object of the present invention is to provide a moisture meter in which a heat-illness risk can be detected early and which is effective as an assistance means for a subject to carry out a proper moisture adjustment. A second object of the present invention is to provide a structure to be measured easily in a body moisture meter in which an armpit is made to be a measurement region.

A third object of the present invention is to obtain a situation in which it can be judged easily whether or not it is in a dehydrated state for a body moisture meter in which an armpit is made to be a measurement region.

The moisture meter of the present invention is a moisture meter measuring moisture content of a subject and is characterized by including: a moisture measurement unit, held by an armpit of the subject, for measuring the amount of moisture of the subject by being in contact with the skin surface of the armpit; a sensor unit for measuring the temperature and humidity of the environment of the subject; and a processing unit which obtains the amount of moisture of the subject from the moisture measurement unit, which sets a Wet-Bulb Globe temperature (WBGT) value from the relation between the temperature and the humidity from the sensor unit, and which determines risk-index of heat illness by referring to the relation table between the amount of moisture of the subject and the Wet-Bulb Globe temperature (WBGT) value.

According to the aforesaid constitution, since a heat illness makes progress when dehydration makes progress under a hot environment, there is obtained a situation in which a subject can comprehend the degree of the heat illness when measuring the amount of moisture and in which it becomes possible for the subject to carry out a proper moisture adjustment. More specifically, it is possible to provide a moisture meter in which it is possible to detect a heat-illness risk-index early and which is effective as an assistance means for a subject to carry out a proper moisture adjustment. More specifically, a Wet-Bulb Globe temperature (WBGT) value is to be set from a relation between the amount of moisture of the subject, which is a moisture-intake situation of the subject, and the temperature and humidity of the environment, which indicate the outside environment, and then, the heat-illness risk-index (degree of heat-illness risk) is to be judged with reference to a relation table between the amount of moisture of the subject and the Wet-Bulb Globe temperature (WBGT) value, so that it is possible for the subject to detect the heat-illness risk early and for the subject to carry out a proper moisture adjustment.

Preferably, the moisture meter is characterized by including: a main body unit; a measurement-unit holding unit which is disposed at one end of the main body unit, which holds the moisture measurement unit, and which is sandwiched by the armpit; a display-unit holding unit which is disposed at the other end of the main body unit and which holds a display unit that displays the measured amount of moisture of the subject and the heat-illness risk-index, wherein the sensor unit is connected to the other end of the main body unit through electrical wiring.

According to the aforesaid constitution, it is possible for the sensor unit to be located, through the electrical wiring, at a position apart from the other end of the main body unit, which is apart from the measurement-unit holding unit, so that it is possible for the sensor unit to measure the air-temperature and humidity of the environment without being affected by the body-temperature of the subject at a position apart from the subject as much as possible.

Preferably, the moisture meter is characterized by including: a main body unit; a measurement-unit holding unit which is disposed at one end of the main body unit, which holds the moisture measurement unit, and which is sandwiched by the armpit; a display-unit holding unit which is disposed at the other end of the main body unit and which holds a display unit that displays the measured amount of moisture of the subject and the heat-illness risk-index, wherein the sensor unit is directly provided at the other end of the main body unit.

According to the aforesaid constitution, it is possible for the sensor unit to be located directly at the other end of the main body unit, which is apart from the measurement-unit holding unit, so that it is possible for the sensor unit to measure the air-temperature and humidity of the environment without being affected by the body-temperature of the subject at a position apart from the subject as much as possible.

Preferably, the moisture meter is characterized in that the measurement-unit holding unit comprises a body-temperature measuring unit for measuring the body-temperature of the subject.

According to the aforesaid constitution, it is possible for the measurement-unit holding unit to measure the amount of moisture of the subject by the moisture measurement unit and to measure the subject body-temperature concurrently. Preferably, the moisture meter is characterized in that there is employed a constitution in which it is possible for the display unit to display the body-temperature of the subject and the Wet-Bulb Globe temperature (WBGT) value other than the amount of moisture of the subject and the heat-illness risk-index.

According to the aforesaid constitution, it is possible for a subject to visually confirm the body-temperature of the subject and the Wet-Bulb Globe temperature (WBGT) value other than the amount of moisture of the subject and the heat-illness risk-index only by viewing the display unit.

Also, in the present invention, the moisture meter is characterized by including: a main body unit formed in a linear shape; a sensor unit which measures data relating to moisture inside a living body by being in contact with a body surface of a subject; and an insertion unit which holds the sensor unit at its distal surface slidably toward a direction approximately perpendicular to the distal surface and also which outputs a signal for instructing the measurement-start of the sensor unit by detecting the slide of the sensor unit, wherein for the housing of the insertion unit, the distal surface is formed such that the angle formed between the longitudinal direction of the main body unit and the slide direction of the sensor unit becomes approximately 20° to 45°, and also, is formed so as to go along the slide direction in the vicinity of the distal surface.

Preferably, the moisture meter is characterized in that the lower surface of the housing of the insertion unit is formed by being curved toward the distal surface. Preferably, the moisture meter is characterized in that the length of the insertion unit is defined such that the distance from the boundary position between the main body unit and the insertion unit to the sensor unit will become 40 mm to 90 mm.

Preferably, the moisture meter is characterized in that the insertion unit is constituted such that the cross-section area thereof becomes small toward the distal surface.

Further, in the present invention, the moisture meter is characterized by including: a sensor unit which outputs a signal relating to the amount of moisture inside a living body by being in contact with a body surface of an armpit of a subject; a conversion means which converts the signal from the sensor unit to the amount of body water; a display means which displays the amount of body water obtained by the conversion means; and a changing means which changes the display mode by the display means so as to call users' attention in a case in which the amount of body water obtained by the conversion means is lower than a first reference value, wherein the first reference value is a value corresponding to a predetermined value between 25% to 40% in a case in which signals outputted when the sensor unit measures water and when it measures air are allotted to 100% and 0% amounts of body water respectively in which the signal outputted by the sensor unit and the amount of body water are correlated by a linear relation.

Preferably, the moisture meter is characterized in that the predetermined value is 35%.

Preferably, the moisture meter is characterized in that the changing means changes the display mode by the display means to still another mode in a case in which the amount of body water obtained from the conversion means is lower than a second reference value and the second reference value is a value smaller than the predetermined value.

Preferably, the moisture meter is characterized in that the second reference value is 25%.

Preferably, the moisture meter is characterized in that the conversion means sets a value corresponding to a predetermined value between 35% to 25% in a case in which signals outputted when the sensor unit measures water and when it measures air are allotted to 100% and 0% amounts of water respectively in which the signal outputted by the sensor unit and the amount of water are correlated by a linear relation.

Also, in a display control method of a body moisture meter including a sensor unit which outputs a signal relating to the amount of moisture inside a living body by being in contact with a body surface of an armpit of a subject, the present invention is characterized by a body moisture meter including: a conversion process which converts the signal from the sensor unit to the amount of body water; a display process which displays the amount of body water obtained in the conversion process; and a changing process which changes the display mode on the display unit so as to call users' attention in a case in which the amount of body water obtained in the conversion process is lower than a first reference value, wherein the first reference value is a value corresponding to a predetermined value between 25% to 40% in a case in which signals outputted when the sensor unit measures water and when it measures air are allotted to 100% and 0% amounts of body water respectively in which the signal outputted by the sensor unit and the amount of body water are correlated by a linear relation.

### BRIEF DESCRIPTION OF DRAWINGS

FIG. 1 is an illustration of a state in which a subject is using a first embodiment of a moisture meter representing one example of the moisture meter disclosed here.

FIGS. 2A and 2B are front-face side and the upper side views respectively of the outward-appearance of the moisture meter shown in FIG. 1.

FIG. 3 is a block diagram showing features of the moisture meter shown in FIGS. 2A and 2B.

FIG. 4 is a diagram showing a structural example of an electrode unit of an impedance-type moisture measurement unit constituting part of the moisture meter shown in FIG. 3.

FIG. 5 is an illustration of a mutual-relation example between the amount of moisture of a living body of a subject M and the body-temperature of the living body of the subject M.

FIG. 6 is a diagram showing an example of a relation table among the WBGT-value (WBGT-temperature), the air-temperature, and also, the relative humidity in which the vertical axis expresses air-temperature (° C.) (dry-bulb temperature) and the horizontal axis expresses relative humidity (%).

FIG. 7 is a diagram showing an example of a heat-illness risk-judgment table which is referred to when obtaining risk-index of heat illness in the moisture meter shown in FIGS. 1 to 3.

FIG. 8 is an operational flow chart showing an operational example of a moisture meter.

FIGS. 9A and 9B are front-face side and the upper side views respectively of the outward-appearance of a second embodiment of the moisture meter representing another example of the moisture meter disclosed here.

FIGS. 10A and 10B are front-face side and the upper side views respectively of the outward-appearance of a third embodiment of the moisture meter representing a further example of the moisture meter disclosed here.

FIG. 11 is a block diagram showing features of the moisture meter shown in FIGS. 10A and 10B.

FIG. 12 is a diagram showing a structural example of a moisture measurement unit in the embodiment of the moisture meter shown in FIG. 11.

FIG. 13 is a side view showing an outward-appearance of a body moisture meter according to a fourth embodiment representing a further example of the moisture meter disclosed here.

FIG. 14 is a side view explaining the housing shape of the body moisture meter.

FIGS. 15A and 15B are illustrations explaining a manner of usage of a body moisture meter disclosed here, in which FIG. 15A illustrates the upper portion of the measurer's or user's arm, while FIG. 15B is the cross-section through the section line 15B-15B in FIG. 15A.

FIG. 16 is a block diagram showing features of the moisture meter shown in FIG. 13.

FIG. 17 is a diagram explaining a measurement circuit of the body moisture meter.

FIG. 18 is an operational flow chart showing an operational example of the moisture meter shown in FIG. 13.

FIG. 19 is a diagram showing a data structure of measurement information.

FIG. 20 is a side view showing an outward-appearance of a body moisture meter according to a fifth embodiment representing a further example of the moisture meter disclosed here.

FIG. 21 is a side view showing an outward-appearance of a body moisture meter according to a sixth embodiment representing a further example of the moisture meter disclosed here.

FIG. 22 is an operational flow chart explaining operational aspects of the body moisture meter shown in FIG. 21.

FIGS. 23A and 23B are diagrams explaining one example of a calibration method of a body moisture meter.

### DETAILED DESCRIPTION

The following description of embodiments of the moisture meter should be understood to be a description of examples of the moisture meter disclosed here, but the scope of the invention is not limited by the disclosed embodiments and features of the disclosed examples of the moisture meter.

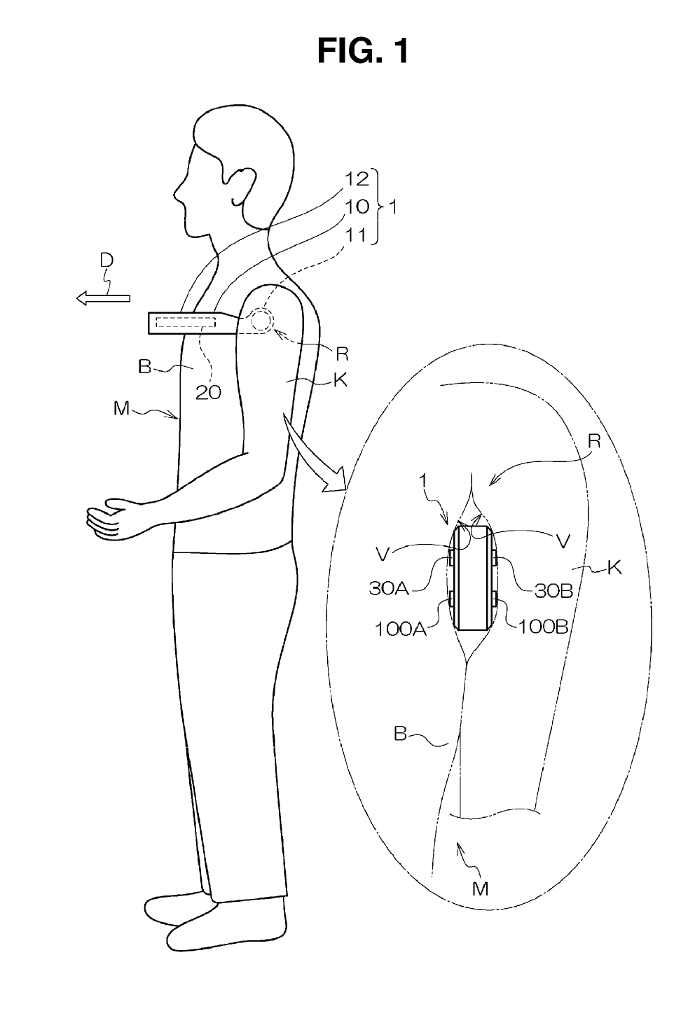

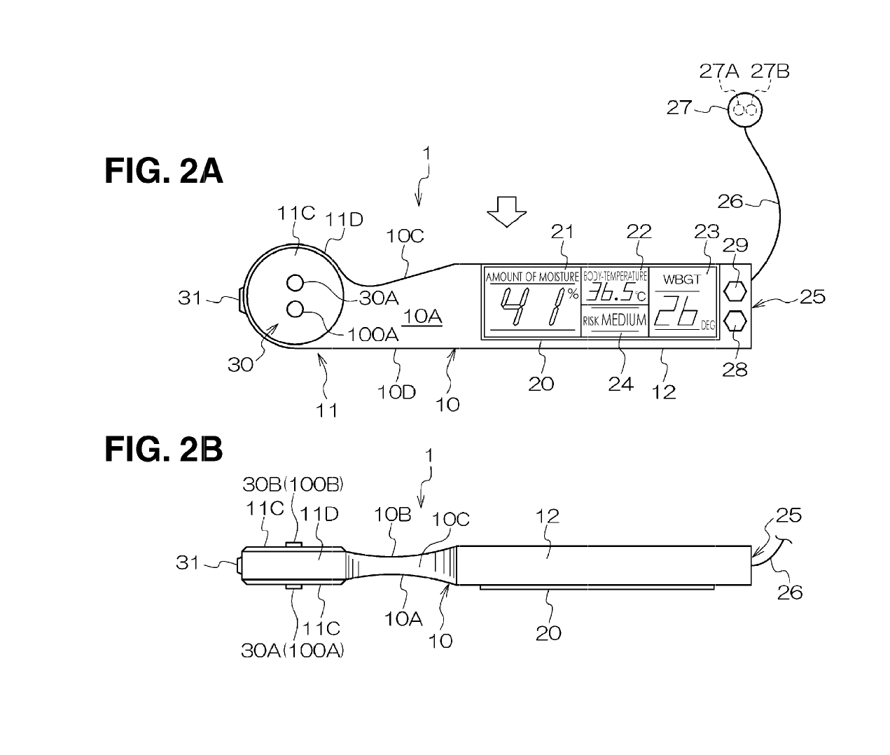

FIGS. 1, 2A and 2B illustrate an embodiment of a moisture meter representing one example of the disclosed moisture meter. The moisture meter 1, also referred to as an electronic moisture meter or an armpit-type electronic moisture meter, is a moisture meter which is relatively small-sized and portable. As shown in FIG. 2, the moisture meter 1 generally includes a main body unit 10, a measurement-unit holding unit 11 and a display-unit holding unit 12, in which the whole weight of the moisture meter 1 is designed to be lightweight so as not to drop or fall even when being sandwiched in an armpit R by a subject (measurer) M as shown in FIG. 1.

The measurement-unit holding unit 11 is provided on one end portion of the main body unit 10 and the display-unit holding unit 12 is provided on the other end portion of the main body unit 10. The main body unit 10 is made, for example, by plastic.

Approximately a mid portion of the main body unit 10 possesses a shape which is relatively easily grasped by the hand of the user or subject M in FIG. 1 and which is also configured or formed so as to be sandwiched relatively easily into the armpit R of the user or subject. In the example shown in FIGS. 2A and 2b, the main body unit 10 includes a first curved front-face portion 10A which is curved loosely toward the inside; a second curved rear-surface portion 10B, on the opposite side, which is gently curved toward the inside; a curve-shaped side-face portion 10C, on the upper side, which is curved loosely toward the inside; and a linear side-face portion 10D on the lower side. The first curved front-face portion 10A and the second curved rear-surface portion 10B curve towards one another to define a narrowed region as seen from the side.

The reason the main body unit 10 is shaped or configured in this manner is to provide a configuration that makes the subject M, who holds or grasps the main body unit 10 by hand, sandwich the measurement-unit holding unit 11 of the moisture meter 1 into the armpit R of FIG. 1 so as to be held reliably. The fact that the amount of moisture of the living body of the subject M is measured in this manner by using the moisture meter 1 and by selecting the armpit R as a region of a living body in which the amount of moisture of the subject M can be measured properly is due to the following reason. The reason the amount of moisture is measured at the armpit R is because the moisture state of the whole living body of the subject M is accurately reflected by the moisture state or level in the armpit. And even with regard to, for example, an aged and thin person, it is possible for the measurement-unit holding unit 11 of the moisture meter 1 to be sandwiched and held reliably by the armpit R between the body and the upper arm. In addition, even if the subject is an infant, if the armpit R is selected, it is possible for the measurement-unit holding unit 11 to be sandwiched relatively easily and held reliably.

As shown in FIG. 2, the measurement-unit holding unit 11 of the moisture meter 1 which is positioned in the armpit of the subject during moisture measurement includes a circular-shaped outer-circumferential portion 11D, a raised or bulging portion 11C on one face of the unit, a raised or bulging portion 11C on the opposite face of the unit, and with respect to the armpit R of the subject M shown in FIG. 1, if the measurement-unit holding unit 11 is held in a state that the measurement-unit holding unit 11 is sandwiched and is pressed by the upper arm K from the upper side by way of the two raised portions 11C, it is possible to stably measure the amount of moisture and the body-temperature of the living body of the subject M. The one raised or bulging portion 11C is formed on the front-face side of the measurement-unit holding unit 11 and the other side raised or bulging portion 11C is formed on the rear face side of the measurement-unit holding unit 11.

As shown in FIG. 1, in a state in which the measurement-unit holding unit 11 of the moisture meter 1 is held by the armpit R, the moisture meter 1 can be held on the upper body B side of the subject M more reliably due to the fact that the main body unit 10 is closely in contact with the side-face portion of the upper body B of the subject.

For example, as shown in FIG. 1, when using the moisture meter 1, it is possible for the display-unit holding unit 12 to be held approximately horizontally toward the front side D of the subject M. The distance between the measurement-unit holding unit 11 and the display-unit holding unit 12, that is the length of the main body unit 10, is such that when the subject M sandwiches the measurement-unit holding unit 11 into the armpit R, the display unit 20 forming a part of the display-unit holding unit 12 is positioned outside the armpit R so that the display unit 20 is not sandwiched between the body portion and the upper arm K of the subject M, thus allowing the display unit 20 to be viewed by the subject M when the measurement-unit holding unit 11 is positioned in the armpit R, sandwiched between the body portion and the upper arm K of the subject.

The display-unit holding unit 12 shown in FIG. 2 has a rectangular shaped cross-section. A rectangular display unit 20, for example, is disposed on the front-face of the display-unit holding unit 12. It is possible for this display unit 20 to be in the form of, for example, a liquid crystal display device, an organic EL device or the like.

A speaker 29 and a buzzer 28, constituting sound generation units, are disposed on the front-face of the display-unit holding unit 12 at a position adjacent the display unit 20. In this manner, the display unit 20, the speaker 29 and the buzzer 28 are disposed on the front-face of the display-unit holding unit 12, so that there never occurs a situation in which the display unit 20, the speaker 29 and the buzzer 28 will be positioned within the armpit R. It is thus possible for the subject M to visually-observe information such as the amount of moisture, the body-temperature or the like which is displayed on the display unit 20 reliably and to hear a sound guidance or the like which generated from the speaker 29, and the buzzer 28 can generate a sound for a necessary warning which can also be heard by the subject.

However, it is possible for the buzzer 28 to be provided at an arbitrary position and it is also possible for the buzzer 28 not to be provided.

As shown in FIG. 2A, the display unit 20 includes, for example, a display screen 21 displaying the amount of moisture (%) inside the living body of the subject (hereinafter, referred to as display screen of amount of moisture), a display screen 22 displaying the body-temperature (° C.) (hereinafter, referred to as display screen of body-temperature), a WBGT-index display unit 23 (displayed by “degree” or “° C.”) which will be explained later, and a heat-illness risk-index display unit 24. The WBGT-index (Wet-Bulb Globe Temperature (unit: ° C.)) is referred to also as a WBGT-value and this WBGT-index will be explained later.

In the example shown in FIG. 2A, the display screen 21 of the amount of moisture in the display unit 20 can display the value of the amount of moisture, for example, as 41% or the like by a relatively large sized digital display. It is possible for the display screen 22 of the body-temperature to display the body-temperature (° C.) of the subject by a digital display of the body-temperature which is displayed in a smaller size compared with the digital display of the amount of moisture. It is possible for the WBGT-index display unit 23 to carry out a digital display by a size as large as that of the amount of moisture display screen 21 and it is possible for the heat-illness risk display unit 24 to display the heat-illness risk-index (degree of heat-illness risk) in a manner of, for example, three steps of displays such as “small”, “medium”, “large” or the like. The display unit 20 is an example of display means for displaying the amount of body moisture.

Thus, it is possible for the subject to confirm the body-temperature and the heat-illness risk index of the subject other than the amount of moisture of the subject and the Wet-Bulb Globe temperature (WBGT) value by visual observation simply by viewing the display unit 20 at the time of measurement, so that it is convenient when using the moisture meter 1.

At an end portion 25 of the display-unit holding unit 12 of the main body unit 10, a sensor unit 27 for measuring the WBGT-index (value) is connected separately by way of an electrical wiring 26. The inside of the sensor unit 27 houses a temperature meter 27A and a humidity meter 27B. Thus, it is possible for the sensor unit 27 to be positioned separately from, and movable relative to, the other end of the main body unit 10 through the electrical wiring 26 so that the sensor unit 27 is spaced apart from the measurement-unit holding unit 11. It is thus possible for the sensor unit 27 to measure the environment air-temperature and humidity without being affected by the body-temperature of the subject M by being at a position spaced apart as much as possible from the body of the subject M.

As shown in FIGS. 2A and 2B, the measurement-unit holding unit 11 of the moisture meter 1 keeps or houses a moisture measurement unit 30 of a so-called bioelectrical impedance type (hereinafter, referred to as impedance type) and a body-temperature measuring unit 31. It is preferable for the surface of the measurement-unit holding unit 11 to be provided with an antislip means by providing concavity and convexity, for example, by a dimple process or the like. Thus, in a case in which the subject M sandwiches the measurement-unit holding unit 11 into the armpit R, there is obtained a shape which can sandwich the measurement-unit holding unit 11 of the moisture meter 1 reliably and stably, and concurrently, the thermal capacity is reduced and it is possible to reach a thermal equilibrium state early.

The impedance-type moisture measurement unit 30 shown in FIGS. 2A and 2B is a portion for measuring, in the armpit R of the subject shown in FIG. 1, the amount of moisture of the living body of the subject M using bioelectrical impedance. As shown in FIG. 1 and FIGS. 2A and 2B, a first electrode unit 30A for supplying measurement electric-current and a first electrode unit 100A for electric-potential measurement are preferably provided at the one raised portion 11C on the one face or side of the measurement-unit holding unit 11, and a second electrode unit 30B for supplying measurement electric-current and a second electrode unit 100B for electric-potential measurement are preferably provided at the other raised portion 11C on the other face or side of the measurement-unit holding unit 11.

For example, as shown FIG. 1, when the impedance-type moisture measurement unit 30 is positioned in the armpit R of the subject (sandwiched between the body part B and the upper arm K), the first electrode unit 30A for supplying measurement electric-current and the first electrode unit 100A for electric-potential measurement are closely in contact with a skin surface V on the side-face portion side of the upper body B, and the second electrode unit 30B for supplying measurement electric-current and the second electrode unit 100B for electric-potential measurement are closely in contact with the skin surface V on the inner surface side of the upper arm K.

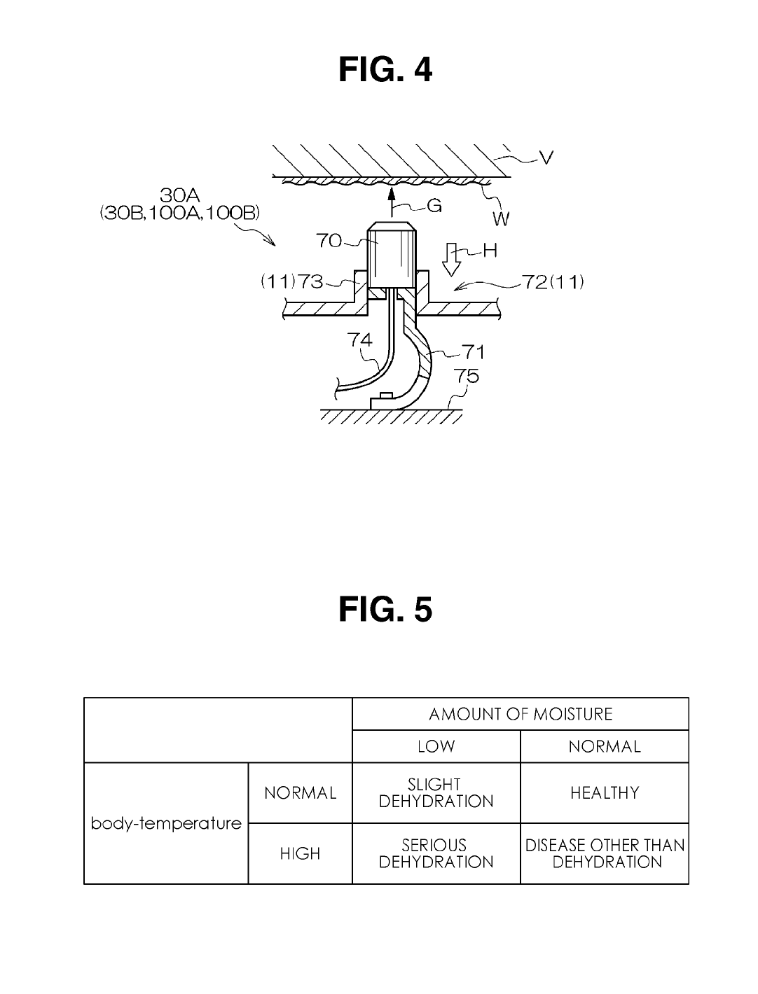

Thus, as shown in FIG. 1, the first electrode unit 30A for supplying measurement electric-current, the first electrode unit 100A for electric-potential measurement, the second electrode unit 30B for supplying measurement electric-current and the second electrode unit 100B for electric-potential measurement are configured and positioned to measure the amount of moisture of the subject M caused by the fact that it is possible for them to be in contact reliably and directly with the skin surface V of the armpit R. An example of the structural configuration of the first electrode unit 30A for supplying measurement electric-current, the second electrode unit 30B for supplying measurement electric-current, the first electrode unit 100A for electric-potential measurement and the second electrode unit 100B for electric-potential measurement will be explained below with reference to FIG. 4.

Also, the body-temperature measuring unit 31 shown in FIGS. 2A and 2B is a portion for measuring the body-temperature of the living body of the subject M in the armpit R of the subject shown in FIG. 1 and, preferably, is disposed so as to be exposed along the outer-circumferential portion 11D of the measurement-unit holding unit 11. Thus, it is possible for the body-temperature measuring unit 31 to be directly in contact reliably with the skin surface of the armpit R.

The body-temperature measuring unit 31 is configured to detect the body-temperature by being in direct contact with the armpit R (skin) of the subject M shown in FIG. 1 and it is possible for the body-temperature measuring unit 31 to employ, for example, a unit having a thermistor or a unit having a thermocouple. For example, the body-temperature measuring unit 31 is configured or constructed such that the temperature signal detected by the thermistor will be outputted by being converted to a digital signal. This thermistor is, for example, protected in a liquid-tight manner by a stainless-made metal cap. In the measurement-unit holding unit 11, it is possible to measure the amount of moisture of the subject by the moisture measurement unit 30 and concurrently, to measure the body-temperature of the subject M at the same time using the body-temperature measuring unit 31.

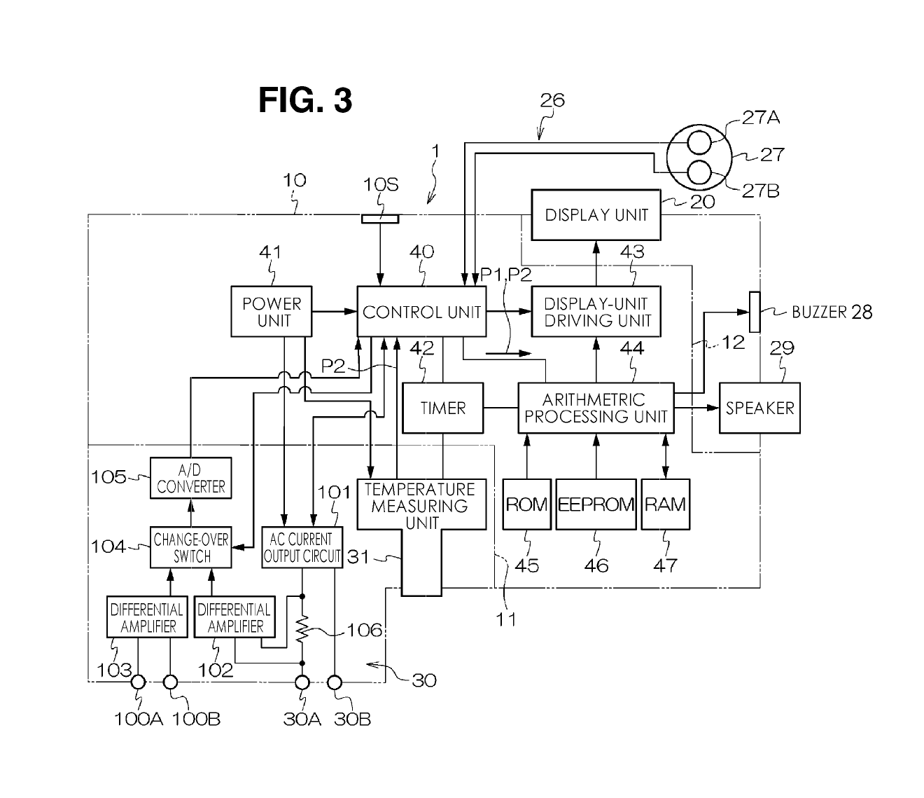

Reference is next made to FIG. 3 which illustrates the features of the moisture meter 1 shown in FIG. 2 providing the functional attributes of the moisture meter 1. In the block of the moisture meter 1 shown in FIG. 3, the main body unit 10 is built-in with a control unit 40, a power unit 41, a timer 42, a display-unit driving unit 43, an arithmetic processing unit (processing unit) 44, a ROM (read only memory) 45, an EEPROM (PROM which can electrically erase and rewrite program contents) 46, and a RAM (random access memory) 47. The impedance-type moisture measurement unit 30 and the body-temperature measuring unit 31 are disposed in the measurement-unit holding unit 11, and the display unit 20, the speaker 29 and the buzzer 28 are disposed in the display-unit holding unit 12.

The power unit 41 in FIG. 3 is a rechargeable secondary battery or a primary battery and supplies power to the control unit 40, the impedance-type moisture measurement unit 30 and the temperature measuring unit 31. The control unit 40 is electrically connected to a power switch 10S, the impedance-type moisture measurement unit 30, the temperature measuring unit 31, the timer 42, the display-unit driving unit 43 and the arithmetic processing unit 44, in which the control unit 40 is constituted so as to control the whole operation of the moisture meter 1. The temperature meter 27A and the humidity meter 27B in the sensor unit 27 are connected electrically to the control unit 40 respectively.

The display unit 20 in FIG. 3 is connected electrically to the display-unit driving unit 43. The display-unit driving unit 43 is constructed so that, as shown in FIGS. 2A and 2B, in response to the command from the control unit 40, the display unit 20 will display, for example, the display screen 21 of the amount of moisture (%) inside the living body of the subject (hereinafter, referred to as display screen of amount of moisture), the display screen 22 of the body-temperature (° C.) (hereinafter, referred to as display screen of body-temperature), the WBGT-index display unit 23 (displayed by “degree”) which will be explained later, and the heat-illness risk display unit 24.

The arithmetic processing unit 44 in FIG. 3 is connected electrically to the speaker 29, the buzzer 28, the ROM 45, the EEPROM 46 and the RAM 47.

Here, there will be explained the impedance-type moisture measurement unit 30 which is shown in FIG. 3.

The following can be said with regard to the measurement of the amount of moisture by a bioelectrical impedance type for the moisture meter 1. A cell tissue of a human body is constituted from a large number of cells and each cell exists in an environment of being filled with extracellular fluid. In the case of flowing electric current to such a cell tissue, a low-frequency AC current mainly flows through an extracellular fluid region and in case of a high-frequency AC current, it flows through an extracellular fluid region and the inside of the cell.

In this manner, the electrical impedance value in the extracellular fluid region in the case of electric current flowing through the cell tissue is composed of only a resistance component and the electrical impedance value of the cell becomes a value obtained by a configuration in which a capacitance component presented by the cell membrane and a resistance component presented by the intracellular fluid are connected in series.

The electrical characteristic of the living body (human body) of the subject M is significantly different depending on the kind of the tissue or the organ. The whole electrical characteristic of the body which includes each of the tissues and the organs like that can be expressed by a bioelectrical impedance.

This bioelectrical-impedance value is a value measured by flowing a minute electric current between a plurality of electrodes which are attached to the subject's body-surface. It is possible from the bioelectrical-impedance value obtained in this manner to estimate the fat percentage, the somatic fat volume, the lean body mass, the amount of body water and the like of the subject (see “Presumption of Moisture Distribution of a Limb by Impedance Method and Use Application thereof”, Medical Electronics and Biological Engineering, vol. 23, No. 6, 1985).

With regard to the amount of moisture inside the living body, there has been known a method in which the amount is presumed by calculating extracellular fluid resistance and intracellular fluid resistance. With regard to the measurement of the amount of moisture, the bioelectrical-impedance value exhibits a relatively low value when the amount of moisture inside the living body is relatively a lot, and the bioelectrical-impedance value exhibits a relatively high value when the amount of moisture inside the living body is relatively little. There is thus known a method of presuming the amount of moisture by calculating the extracellular fluid resistance and the intracellular fluid resistance.

The impedance-type moisture measurement unit 30 which is shown in FIG. 3 is an apparatus for measuring the bioelectrical-impedance value by applying an AC current to the living body of the subject M.

The impedance-type moisture measurement unit 30 shown in FIG. 3 includes the electrode unit 30A for supplying the first measurement electric-current and the electrode unit 30B for supplying the second measurement electric-current; the electrode unit 100A for the first electric-potential measurement and the electrode unit 100B for the second electric-potential measurement; an AC current output circuit 101; two differential amplifiers 102, 103; a change-over switch 104; an A/D converter 105; and a reference resistor 106.

The electrode unit 30A for supplying the first measurement electric-current, the electrode unit 30B for supplying the second measurement electric-current, the electrode unit 100A for the first electric-potential measurement and the electrode unit 100B for the second electric-potential measurement are provided, for example, by being exposed toward the outside (i.e., are not covered) in the measurement-unit holding unit 11 shown in FIG. 2. Thus, by virtue of being exposed, it is possible for these four electrode units 30A, 30B, 100A and 100B to be in contact directly with the skin surface of the armpit R of the subject M shown in FIG. 1.

The AC current output circuit 101 in FIG. 3 is connected electrically to a control unit 40, the electrode unit 30A for supplying the first measurement electric-current and the electrode unit 30B for supplying the second measurement electric-current, and there is disposed the reference resistor 106 between the AC current output circuit 101 and the electrode unit 30A for supplying the first measurement electric-current. The differential amplifier 102 is connected to both the end portions of this reference resistor 106. Another differential amplifier 103 is connected electrically to the electrode unit 100A for the first electric-potential measurement and the electrode unit 100B for the second electric-potential measurement. Two of the differential amplifiers 102, 103 are connected electrically to a control unit 49 through the change-over switch 104 and the A/D converter 105.

In FIG. 3, when the control unit 40 supplies a predetermined application signal for the living body to the AC current output circuit 101, the AC power-supply output circuit 101 supplies AC measurement currents with respect to the first electrode unit 30A for supplying measurement electric-current through the reference resistor 106 and with respect to the second electrode unit 30B for supplying measurement electric-current. The one differential amplifier 102 detects electric-potential difference between the both ends of the reference resistor 106. The other differential amplifier 103 detects electric-potential difference between the electrode units 100A and 100B for electric-potential measurements. The change-over switch 104 selects either one of the electric-potential difference outputs from the differential amplifiers 102, 103 and transmits it to the A/D converter 105, and the A/D converter 105 analogue-to-digital converts the electric-potential difference outputs of the differential amplifiers 102, 103 and supplies them to the control unit 40.

Next, with reference to FIG. 4, there will be explained a structural example of the first electrode unit 30A for supplying measurement electric-current, the second electrode unit 30B for supplying measurement electric-current, the first electrode unit 100A for electric-potential measurement and the second electrode unit 100B for electric-potential measurement of the impedance-type moisture measurement unit 30 discussed above.

With regard to the structure of the first electrode unit 30A for supplying measurement electric-current and the second electrode unit 30B for supplying measurement electric-current and with regard to the structures of the first electrode unit 100A for electric-potential measurement and the second electrode unit 100B for electric-potential measurement, it is possible to employ the same structure respectively. In FIG. 4, there are shown the skin surface V and moisture W existing at this skin surface V.

Each of the structure of the first electrode unit 30A for supplying measurement electric-current, the second electrode unit 30B for supplying measurement electric-current, the first electrode unit 100A for electric-potential measurement and the second electrode unit 100B for electric-potential measurement, which are shown in FIG. 4, includes an electrode terminal 70, an elastic deformation member 71 having a semicircular plate shape and an electrode-terminal guide unit 72. The electrode terminal 70 having electric conductivity is connected to an electrical wiring 74, one end portion of the elastic deformation member 71 is fixed at the bottom portion of the electrode terminal 70 and the other end portion of the elastic deformation member 71 is fixed at a fixed portion 75 inside the measurement-unit holding unit 11 of FIG. 2. The electrode-terminal guide unit 72 includes a tube-shaped unit 73 and a lower portion of the electrode terminal 70 is inserted into or positioned in the tube-shaped unit 73. Thus, when the distal portion of the electrode terminal 70 is pressed against the skin surface V in an arrow G direction, the electrode terminal 70 is pressed in an arrow H direction against the elastic force of the elastic deformation member 71, so that it is possible for the distal portion of the electrode terminal 70 to be in reliable contact with the skin surface V such that the electrode terminal 70 is not spaced apart from the skin surface V.

It is possible for the structure of each electrode unit discussed above to have a construction other than that shown in FIG. 4.

Meanwhile, it is known that when a dehydrated state of the subject M continues, various symptoms as mentioned above progress. Amongst those symptoms, heat illness can be a big problem. For a method of early detecting the heat illness occurring by the dehydrated state or for a method of judging the degree of seriousness of the heat illness, it is desirable to measure the amount of moisture of the subject M in FIG. 1 and concurrently, to measure the body-temperature of the subject M. Based on the mutual relationship between the amount of moisture of the living body of the subject M and the body-temperature of the living body of the subject M, it is possible to judge, for example, as follows for the symptoms of the subject, which will be explained with reference to FIG. 5.

The example of the mutual-relationship between the amount of moisture of the living body of the subject M and the body-temperature of the living body of the subject M, which is shown in FIG. 5, is stored beforehand (i.e., before the meter is first used), for example, in the EEPROM 46 of FIG. 3. In FIG. 5, in a case in which the amount of moisture is relatively low and if the body-temperature has a normal value, the subject suffers from a slight dehydration, and in a case in which the amount of moisture is normal and the body-temperature is normal, the subject is in a healthy condition. On the other hand, in a case in which the amount of moisture is relatively low and if the body-temperature is high, the subject suffers from a serious dehydration, and in a case in which the amount of moisture is normal and the body-temperature is relatively high, it can be said that the subject suffers from a disease such as a flu other than the dehydration.

In this manner, it becomes possible, based on the amount of moisture and the body-temperature of the living body of the subject, to judge the health of the subject, slight and serious dehydrations, and a flu-symptom, so that according to the moisture meter 1 disclosed here by way of examples, the measurement of the amount of moisture and the measurement of the body-temperature in the armpit R are important.

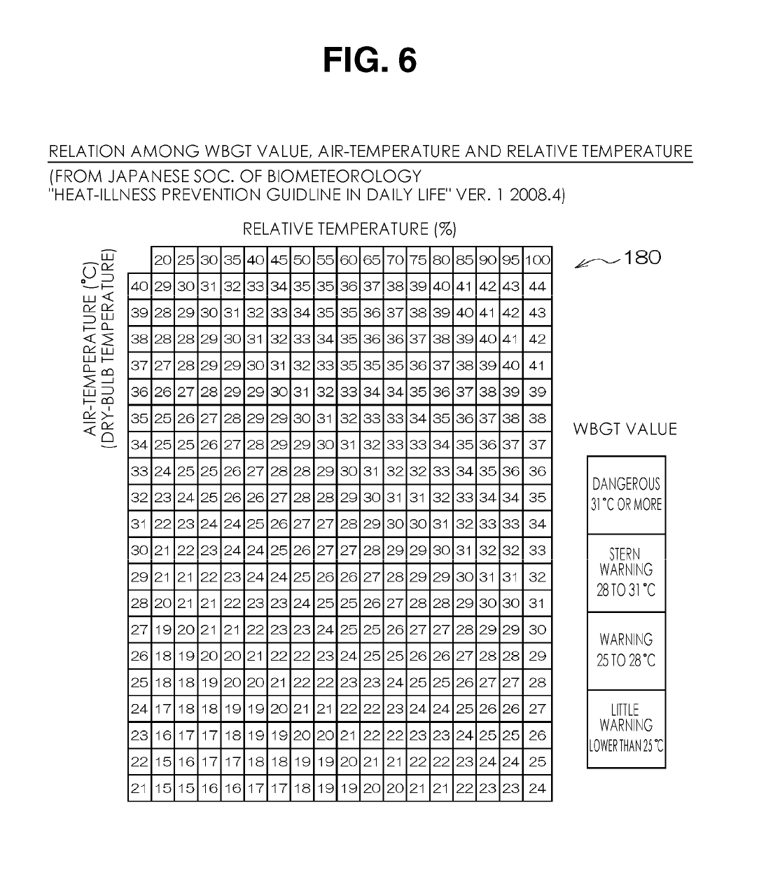

As already explained, when dehydration progresses, heat illness can occur. To judge the degree of the heat-illness risk, there will be explained the WBGT-index (WBGT-value) mentioned above with reference to FIG. 6.

In FIG. 6, the vertical axis indicates air-temperature (° C.) (dry-bulb temperature) and the horizontal axis indicates relative humidity (%) in which there is shown a WBGT-value table 180 which indicates a relation example among the WBGT-value (WBGT-temperature), air-temperature and relative humidity and which is presented from the source: ““Heat-Illness Prevention Guideline in Daily Life” by Japanese Soc. of Biometeorology, Ver. 1, 2008.4”.

In the WBGT-value table 180 shown in FIG. 6, for example, if the WBGT-value is 31 degrees or more, this shows that the degree of heat-illness risk is characterized as “dangerous”, if the WBGT-value is between 28 degrees and 31 degrees, this shows that the degree of heat-illness risk is characterized as “stern warning”, if the WBGT-value is between 25 degrees and 28 degrees, this shows that the degree of heat-illness risk is characterized as “warning”, and then, if the WBGT-value is less than 25 degrees, this shows that the degree of heat-illness risk is characterized as “little warning”. In FIG. 6, if, for example, the air-temperature is 30° C. and the relative humidity is 90%, the WBGT-value is 32° C. (referred to also as 32 degrees) and this shows that the degree of the heat-illness risk is “dangerous”.

The WBGT-index (WBGT-value) shown in FIG. 6 is a relatively simple and convenient index for carrying out the evaluation of heat stress caused by the hot environment which a worker receives in a labor environment. In case of evaluating the hot environment, it is necessary to make a comprehensive evaluation in consideration of humidity, wind velocity and radiation (emission) heat in addition to air-temperature, in which the WBGT-value becomes a value which is found by synthesizing these basic various hot-fever factors.

The WBGT-value table 180 shown in FIG. 6 is, for example, stored in the EEPROM 46 of FIG. 3.

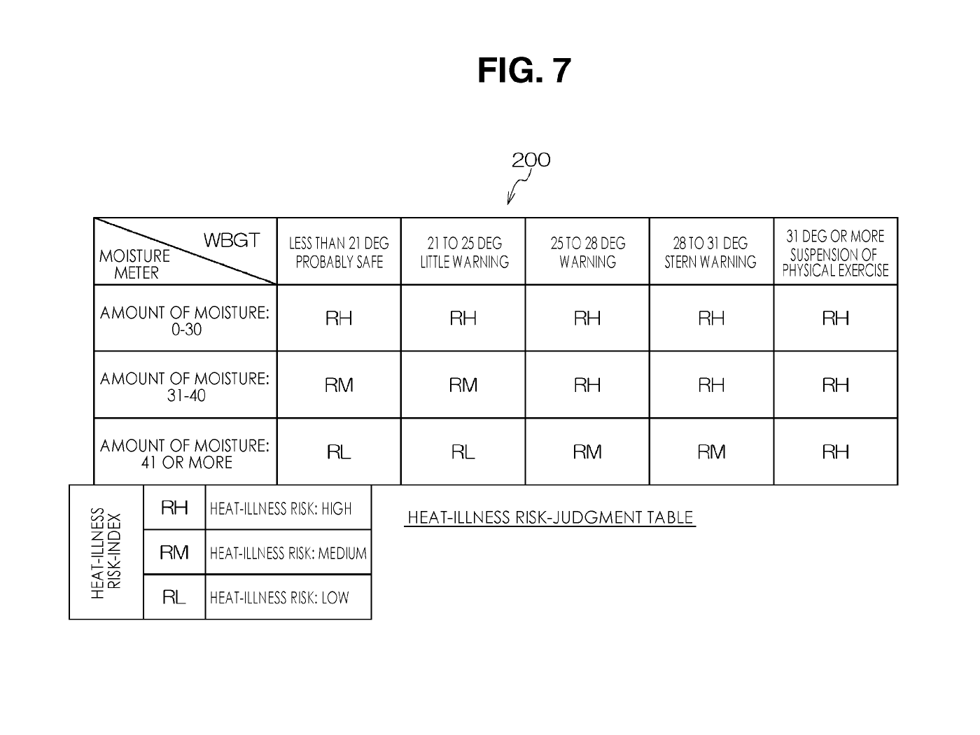

FIG. 7 shows a heat-illness risk-judgment table 200 used for judging the heat-illness risk-index for the moisture meter 1 of the embodiment of the present invention shown in FIGS. 1 to 3 representing one example of the moisture meter disclosed here. The heat-illness risk-judgment table 200 shown in this FIG. 7 is, for example, stored in the EEPROM 46 of FIG. 3.

The vertical axis of the heat-illness risk-judgment table 200 in FIG. 7 represents a classification example of the amount of moisture and the horizontal axis of the table represents a classification example of the WBGT-values.

With regard to the vertical axis of the heat-illness risk-judgment table 200, the amount of moisture is classified according to, for example, three ranges, specifically a range in which the amount of moisture is 0% to 30%, a range in which the amount of moisture is from 31% to 40% and a range in which the amount of moisture is 41% or more.

On the other hand, the WBGT-values are classified according to five ranges, specifically a range in which the degree of heat-illness risk is characterized as “dangerous: this means suspension of physical exercise” if the WBGT-value is 31 degrees or more, a range in which the degree of heat-illness risk is characterized as “stern warning” if the WBGT-value is from 28 to 31 degrees, a range in which the degree of heat-illness risk is characterized as “warning” if the WBGT-value is from 25 to 28 degrees, and then, a range in which the degree of heat-illness risk is under “little warning” if the WBGT-value is from 21 to 25 degrees, and a range in which the degree of the heat-illness risk is characterized as “probably safe” if the WBGT-value is less than 21 degrees.

The heat-illness risk-judgment table 200 of FIG. 7 defines three ranges for classification. That is, a reference numeral RH indicates that the degree of heat-illness risk is “high”, a reference numeral RM indicates that the degree of heat-illness risk is “medium” and a reference numeral RM indicates that the degree of heat-illness risk is “low”.

As already explained, the EEPROM 46 shown in FIG. 3 stores the WBGT-value table 180 shown in FIG. 6, the heat-illness risk-judgment table 200 shown in FIG. 7 and predetermined sound data other than those above.

The ROM 45 shown in FIG. 3 stores data of the amount of moisture, which are obtained from the impedance value measured by the impedance-type moisture measurement unit 30 based on the timing measured by the timer 42, and a program for forecast-calculating the amount of moisture and the body-temperature of a subject based on the time change of the data of the amount of moisture and the body-temperature data, which was calculated from the body-temperature data measured by the temperature measuring unit 31.

In addition, the ROM 45 stores a program for specifying the WBGT-index (WBGT-value) from the WBGT-value table 180 shown in FIG. 6 based on the air-temperature obtained from the temperature meter 27A of the sensor unit 27 shown in FIG. 3 and the relative humidity obtained from the humidity meter 27B of the sensor unit 27.

Further, the ROM 45 stores a program for specifying the degree RH, RM, RL of the heat-illness risk by referring to the amount of moisture of the subject and the WBGT-value, which were obtained, with respect to the heat-illness risk-judgment table 200 shown in FIG. 7.

It is possible for the RAM 47 shown in FIG. 3 to store the calculated data of the amount of moisture and the body-temperature data respectively in time-series. That is, the calculated moisture amount data and the body-temperature data can be stored over a period of time so that changes or trends in the calculated moisture amount data and the body-temperature data can be observed. In addition, it is possible for the RAM 47, as already explained, to store the amount of moisture of the subject and the WBGT-index (WBGT-value) which were obtained.

As already mentioned, it is known in general that when the body-temperature varies, the bioelectrical-impedance value also varies. That is, the amount of moisture varies such that the bioelectrical-impedance value declines when the body-temperature is increased, and the bioelectrical-impedance value is increased when the body-temperature declines. Consequently, it is possible by using the measured body-temperature data to correct the bioelectrical-impedance value.

The arithmetic processing unit 44 as a processing unit in FIG. 3 forecast-calculates the amount of moisture and the body-temperature of the subject in accordance with the program stored in the ROM 45. The arithmetic processing unit 44 specifies the WBGT-index (WBGT-value) from the WBGT-value table 180 shown in FIG. 6 based on the air-temperature obtained from the temperature meter 27A of the sensor unit 27 and the relative humidity obtained from the humidity meter 27B of the sensor unit 27. The arithmetic processing unit 44 specifies the degree RH, RM, RL of heat-illness risk by referring to the amount of moisture of the subject and the WBGT-index (WBGT-value), which were obtained, with respect to the heat-illness risk-judgment table 200 shown in FIG. 7. Further, the arithmetic processing unit 44 carries out the output of sound data to the speaker 29, the operation of ringing the buzzer 28 or the like.

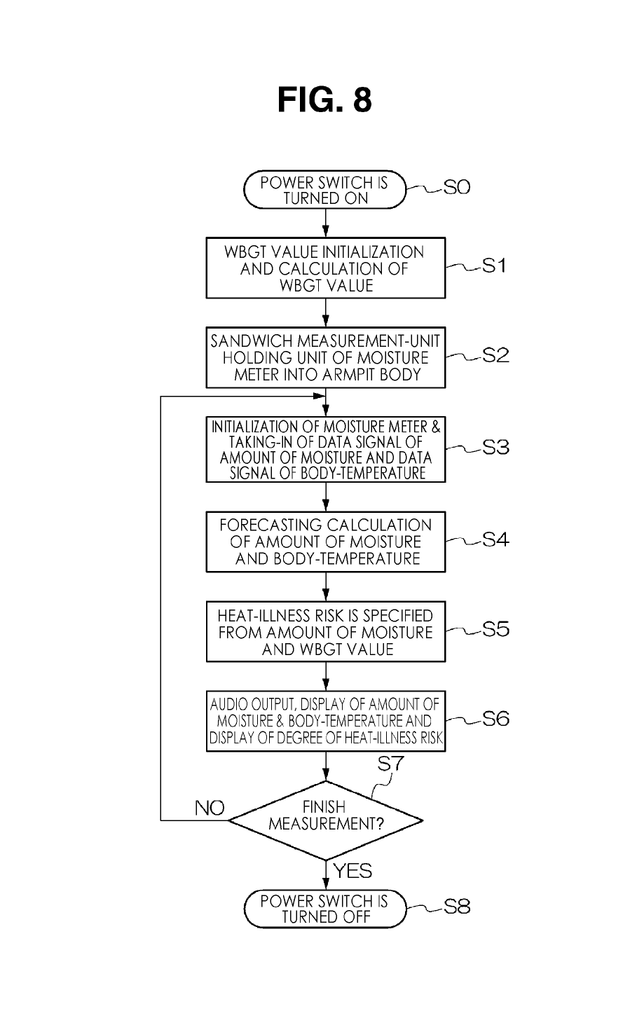

Set forth next, with reference to FIG. 8, is an explanation of an example of usage of the moisture meter 1.

In step S0 in FIG. 8, the subject turns on the power switch 10S shown in FIG. 3 and when the ON signal is transmitted to the control unit 40, the moisture meter 1 will have a measurable state (i.e., the moisture meter is turned on and is in an operational state).

In step S1 in FIG. 8, the control unit 40 in FIG. 3 carries out the initialization of the WBGT-value which was calculated formerly in the arithmetic processing unit 44 and the arithmetic processing unit 44 calculates and specifies the WBGT-index (WBGT-value) from the WBGT-value table 180 shown in FIG. 6 based on the air-temperature obtained from the temperature meter 27A of the sensor unit 27 and the relative humidity obtained from the humidity meter 27B of the sensor unit 27.

Next, in step S2, as shown in FIG. 1, the subject M sandwiches the measurement-unit holding unit 11 of the moisture meter 1 in the armpit R (i.e., between the upper arm K and the upper body B) by using the two raised or bulging portions 11C shown in FIGS. 2A and 2B. That is, the raised or bulging portions allow the measurement-unit holding unit 11 to be rather easily sandwiched in the armpit R. In this manner, in a state in which the measurement-unit holding unit 11 of the moisture meter 1 is held by the armpit R, the moisture meter 1 can be held on the upper body B of the subject more reliably due to the fact that the main body unit 10 is closely in contact with the side-face portion of the upper body B of the subject and, for example, it is possible for the display-unit holding unit 12 to be positioned approximately horizontally toward the front side D of the subject M.

Furthermore, the distance between the measurement-unit holding unit 11 and the display-unit holding unit 12 is set such that in a case in which the subject M sandwiches the measurement-unit holding unit 11 in the armpit R, the display unit 20 will be positioned outside the armpit R (i.e., a position in which the display unit 20 is not sandwiched between the body portion and the upper arm), so that it is possible for the subject M to visually-observe the digital display 24 of the amount of moisture and the digital display 25 of the body-temperature in the display unit 20 of the display-unit holding unit 12 easily. Furthermore, it is possible for the subject M to catch or hear, for example, a sound guidance generated by the speaker 29, a warning sound produced by the buzzer 28 and the like.

In step S3 in FIG. 8, the arithmetic processing unit 44 in FIG. 3 carries out initialization of the moisture meter 1 when the measurement-unit holding unit 11 of the moisture meter 1 is held by the armpit R as shown in FIG. 1 and takes-in data signal P1 from the control unit 40 of the amount of moisture measured through use of the moisture measurement unit 30 and body-temperature data signal P2 measured by the temperature measuring unit 31 at a predetermined sampling time based on the timing signal of the timer 42.

In case of obtaining the data signal P1 of the amount of moisture using the moisture measurement unit 30 in FIG. 3, the first electrode unit 30A for supplying measurement electric-current and the second electrode unit 30B for supplying measurement electric-current, which are in contact with the armpit R of the subject M as shown in FIG. 1, are applied with an AC current from the AC current output circuit 101 with respect to the subject M. Then, the first electrode unit 100A for electric-potential measurement and the second electrode unit 100B for electric-potential measurement, which are in contact with the armpit R of the subject, detect the electric-potential difference between two points in the armpit R of the subject and this electric-potential difference is supplied to the other differential amplifier 103, in which the other differential amplifier 103 outputs the electric-potential difference signal between the two points of the subject M to the change-over switch 104 side.

The one differential amplifier 102 in FIG. 3 outputs the electric-potential difference signal of the reference resistor 106 to the change-over switch 104 side. By the mechanism in which the control unit 40 changes over the change-over switch 104, the electric-potential difference signal from the one differential amplifier 102 and the electric-potential difference signal from the other differential amplifier 103 are analogue-to-digital converted by the A/D converter 105 and supplied to the control unit 40, in which the control unit 40 finds out or determines the bioelectrical-impedance value based on that digital signal. This control unit 40 calculates the data P1 of the amount of moisture from the obtained bioelectrical-impedance value. This data P1 of the amount of moisture is transmitted from the control unit 40 to the arithmetic processing unit 44.

In step S4 in FIG. 8, the arithmetic processing unit 44 forecast-calculates the amount of moisture and the body-temperature of the subject M based on the time change of the data of the amount of moisture and the body-temperature data of the subject, which are obtained from the data P1 of the amount of moisture and the body-temperature data P2 measured by the temperature measuring unit 31. That is, based on data of the body temperature and moisture content in the past and present, it is possible to predict the body temperature and moisture content in the future. It is also possible to predict the future risk of heat stroke.

In step S5, the arithmetic processing unit 44 specifies whether the heat-illness risk is a high degree “RH”, a medium degree “RM” or a low degree “RL” based on the amount of moisture of the subject M and the WBGT-value which were obtained using the heat-illness risk-judgment table 200 shown in FIG. 7.

In step S6 in FIG. 8, the control unit 40 provides a command to the display-unit driving unit 43 when the arithmetic processing unit 44 in FIG. 3 obtains the value of the amount of moisture of the subject M and the degree of the heat-illness risk which were calculated so that, as shown in FIG. 2A, the display unit 20 displays the calculated value of the amount of moisture of the subject M (for example, 41%), the value of the body-temperature (for example, 36.5° C.), the WBGT-index (WBGT-value) (for example, 26 degrees) and the degree of the heat-illness risk (for example, medium: RM). It is possible for the degree of the heat-illness risk and the amount of moisture to be announced to the subject by the speaker 29.

A configuration can be employed in which, for example, the buzzer 28 generates the alarm sound once if the degree of the heat-illness risk is low (RL), generates the alarm sound twice if the degree of the heat-illness risk is medium (RM) and generates the alarm sound three times if the degree of the heat-illness risk is high (RH).

Then, in step S7, in a case in which the subject M finishes the measurement by the moisture meter 1, the power switch 10S in FIG. 3 is turned off in step S8. In the case of not finishing the measurement by the moisture meter 1, the flow returns to step S3 and it becomes a situation in which the processes from step S3 to step S7 are repeated again.

The moisture meter 1 described above as one example of the moisture meter disclosed here is configured such that the amount of moisture of the subject M can be measured in the armpit R through a measurement carried out properly. It is possible for the arithmetic processing unit 44 to forecast-calculate the amount of moisture and the body-temperature of the subject from the bioelectrical-impedance value measured by the first electrode unit 30A for supplying measurement electric-current, the second electrode unit 30B for supplying measurement electric-current, the first electrode unit 100A for electric-potential measurement and the second electrode unit 100B for electric-potential measurement of the impedance-type moisture measurement unit 30 based on time change of the data of the amount of moisture and the body-temperature data of the subject, which can be obtained from the data P1 of the amount of moisture and the body-temperature data P2 measured by the temperature measuring unit 31. Thus, the moisture meter is effective as an assistance means for carrying out a proper moisture adjustment for an infant who has difficulty in his proper drinking-behavior on an occasion of his dry-mouth feeling, for an aged person, for a period during a high-intensity exercise, or the like as well as the moisture adjustment which is very important for the maintenance of health in daily life.

Also, the reason the amount of moisture of the subject M is measured by selecting the armpit as the region of a living body in which the measurement is carried out properly is because the measurement of the amount of moisture in the armpit R is an accurate reflection or indicator of the moisture state of the whole living body of the subject M. Also, in general, the skin of the aged person is dried rather easily and moisture fluctuation depending on persons can be a lot. For those persons, the armpit R has little influence from the outside compared with other regions, so that the armpit is preferable because the measurement fluctuation is relatively little. Even for an aged person who is a thin person, it is possible for the measurement-unit holding unit 11 of the moisture meter 1 to be sandwiched reliably into the armpit R between the body and the upper arm and held in the armpit. Also, the reason for the moisture meter is because even if the subject is an infant, if the armpit R is selected, the measurement-unit holding unit 11 can be sandwiched (positioned) rather easily and reliably held in the armpit. Further, the measurement accuracy is heightened more by employing such a structure in which the moisture measurement unit 30 will maintain a center position within the armpit R.

Furthermore, the moisture meter 1 described above as one example of the moisture meter disclosed here is configured such that the body-temperature in the armpit R can also be measured concurrently when measuring the amount of moisture of the subject M properly. Thus, as shown in FIG. 5, the healthcare worker or the caretaker has only to sandwich and hold the measurement-unit holding unit 11 of the moisture meter 1 in the armpit R of the subject M to measure both the body-temperature and the amount of moisture of the subject M, compared with a case of measuring the body-temperature from an oral and separately measuring the amount of moisture of the subject M.

As shown in FIG. 2A, from the relationship between the amount of moisture of the living body of the subject M and the body-temperature of the living body of the subject M, which are displayed at the display unit 20, the subject is considered to suffer from a slight dehydration if the body-temperature is a normal value in a case in which the amount of moisture is relatively low, and is considered to be in a healthy condition if the body-temperature is normal in a case in which the amount of moisture is normal. On the other hand, it is possible to roughly judge, for example by a medical doctor, that the subject suffers from serious dehydration if the body-temperature is relatively high in a case in which the amount of moisture is relatively low, and the subject is considered to have a flu-symptom if the body-temperature is high in a case in which the amount of moisture is normal.

Furthermore, as also shown in FIG. 2A, it is possible for the display unit 20 of the moisture meter 1 to relatively easily and reliably obtain the degree of heat-illness risk from the relationship between the amount of moisture of the subject and the WBGT-value, which were obtained as described by way of example above, and to display the degree of heat-illness risk. The moisture meter 1 early-detects the risk of heat illness more accurately by comprehending or considering both the moisture-intake situation of the subject and the outside environment, and can thus be used effectively as an assistance means in which the subject can carry out a proper moisture adjustment.

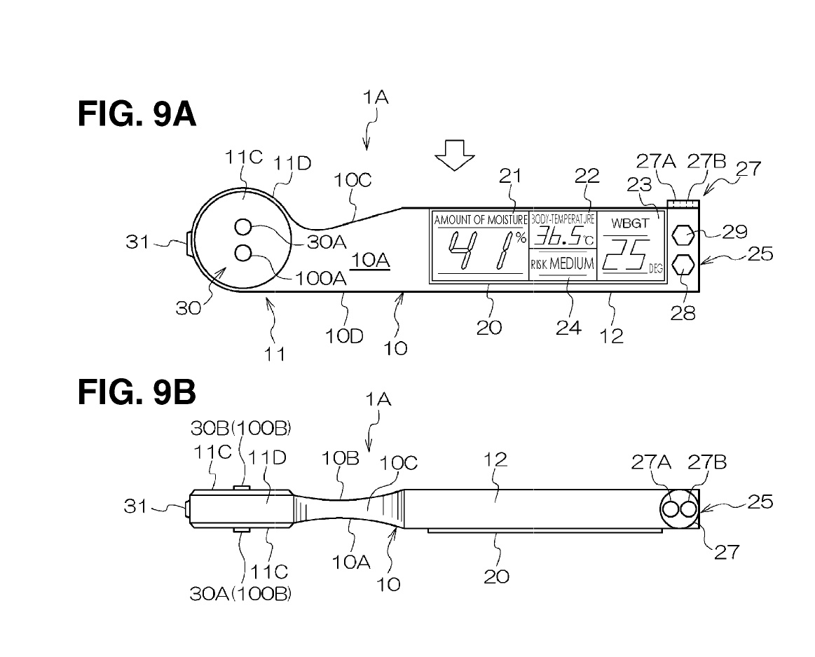

Set forth next with reference to FIGS. 9A and 9B is a description of a second embodiment of the moisture meter, representing another example of the moisture meter disclosed here. Features and aspects of this second embodiment of the moisture meter which are the same as in the first embodiment of the moisture meter are designated by common reference numerals and a detailed description of such features and aspects is not repeated.

The moisture meter 1A shown in FIGS. 9A and 9B includes the sensor unit 27 provided directly at the side-face of the end portion 25 of the display-unit holding unit 12. That is, in this second embodiment, the sensor unit 27 is directly mounted on the end portion 25 of the display-unit holding unit 12 and is not connected to the display-unit holding unit 12 by way of the wiring 26 used in the first embodiment. Thus, the electrical wiring 26 shown in FIGS. 2A and 2B is unnecessary and the handling of the moisture meter 1A is easier. Furthermore, the sensor unit 27 is provided directly at the opposite end of the main body unit from the measurement-unit holding unit 11 so that the sensor unit 27 is spaced apart from the measurement-unit holding unit 11. It is thus possible to measure the air-temperature and humidity of the environment without being affected by the subject body-temperature.

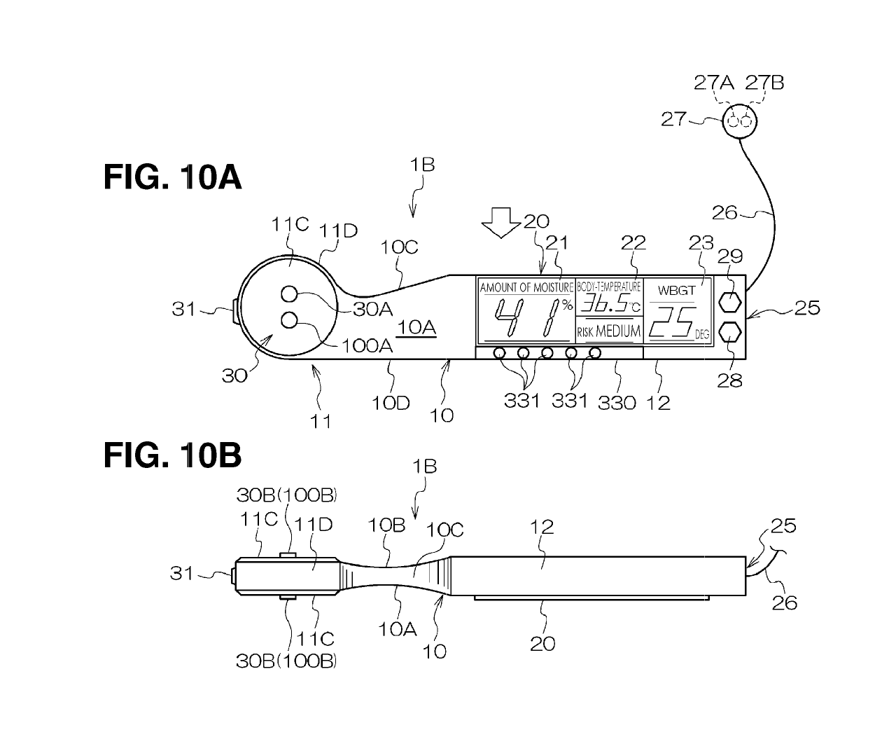

The moisture meter 1B shown in FIGS. 10A and 10B illustrate an alternative configuration in which a temperature display unit 330 in the form of a temperature-sensitive ink is provided at the display-unit holding unit 12. It is possible for this temperature display unit 330 to roughly-display the environment temperature by dot display units 331 having mutually different colors. That is, the dot display units 331 exhibit different colors, and depending on the outside environmental temperature a different one of the dot display units 331 will be visibly colored so that the color displayed by the respective dot display units 331 indicates the outside environmental temperatures.

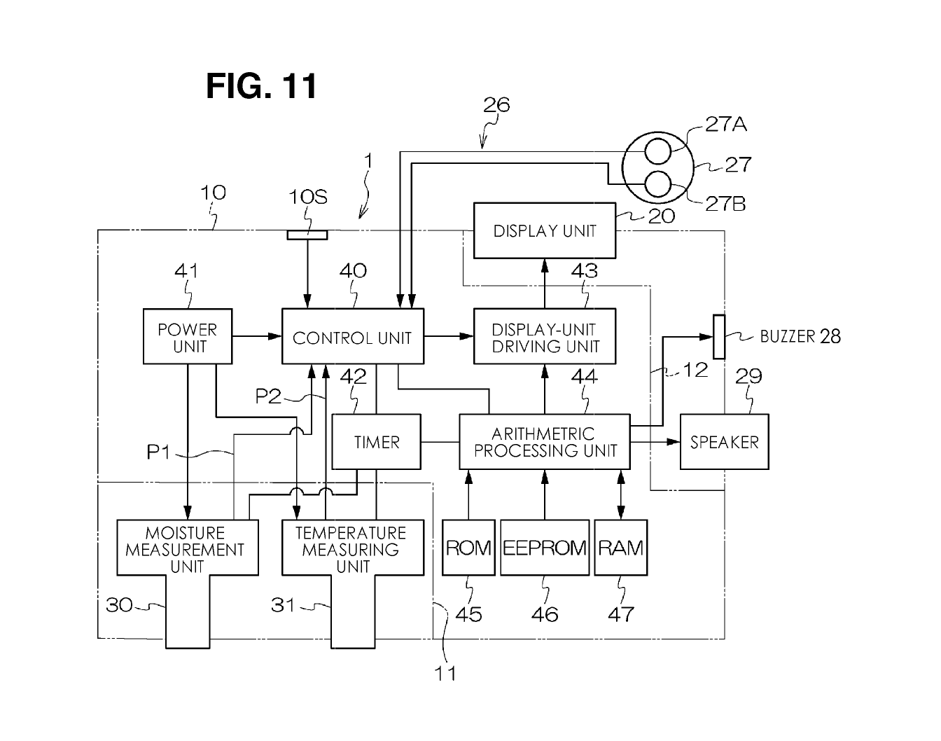

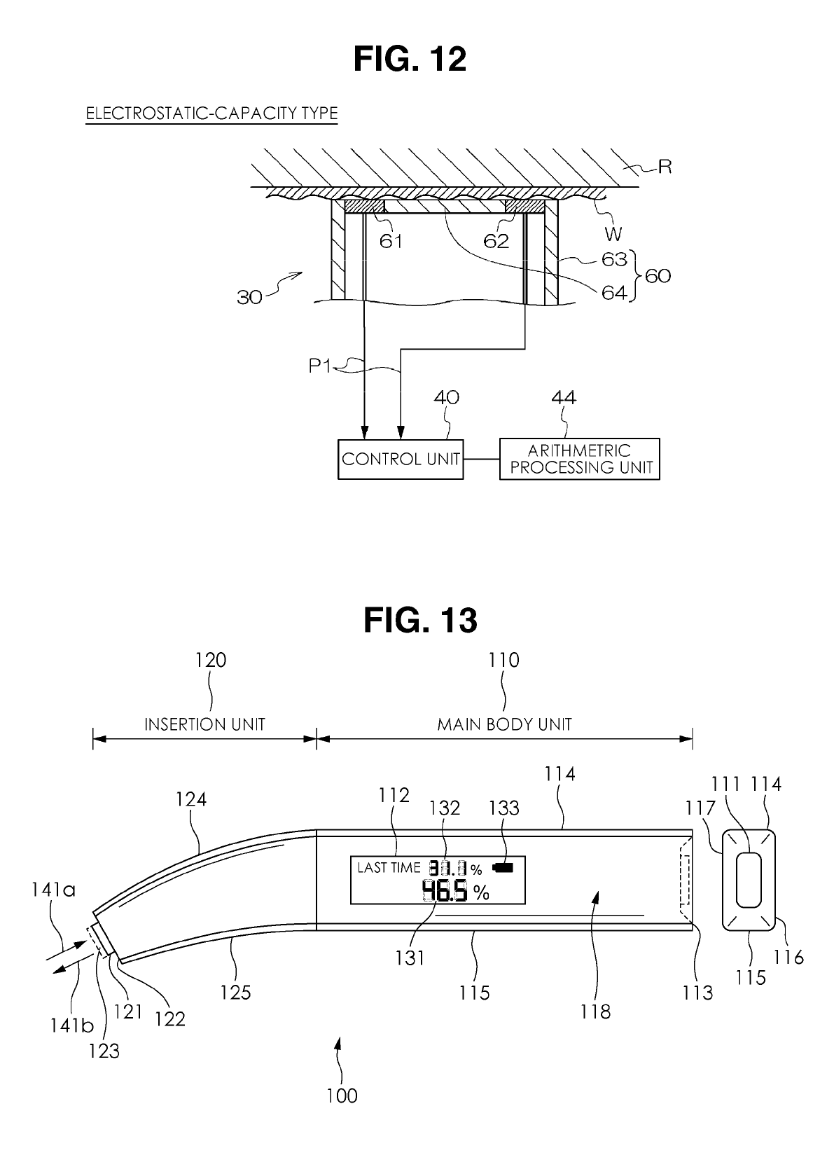

Set forth next, referring to FIGS. 11 and 12, is a description of a third embodiment of the moisture meter, representing another example of the moisture meter disclosed here. The following description focuses on differences between this embodiment and earlier embodiments described above. Features and aspects of this third embodiment of the moisture meter which are the same as in earlier embodiments of the moisture meter are designated by common reference numerals and a detailed description of such features and aspects is not repeated.

The embodiment of the moisture meter shown in FIG. 11 differs in terms of the constitution or construction of the moisture measurement unit 30′ which in the FIG. 11 embodiment is formed as a unit which uses an electrostatic capacity such as shown in FIG. 12. The structure in which the sensor unit 27 is connected electrically to the control unit 40 is the same as in the first embodiment. Hereinafter, explanations for the common portions will be quoted from the explanations of FIG. 3 and the explanation will be carried out by being centered on different points.

The moisture measurement unit 30′ shown in FIG. 11 possesses the construction shown in FIG. 12. More specifically, the electrostatic capacity of the living body of the subject M which is the measurement object is measured and the amount of moisture is determined from the amount of change of the dielectric constant which changes depending on the percentage of moisture content. This moisture measurement unit 30′ includes a container unit 60 and two electrodes 61, 62. The container unit 60 includes a resin-made peripheral portion 63 and a lid portion 64. The two electrodes 61, 62 are disposed at the lid portion 64 so as to be exposed from the lid portion 64 toward the outside in a state of being spaced apart from one another and mutually electrically-isolated.

The two electrodes 61, 62 directly contact the skin of the armpit R and the moisture W on the skin, the electrostatic capacity of the living body of the subject M is measured and the amount of moisture is determined based on the amount of change of the dielectric constant which changes depending on the percentage of moisture content. The data signal from the two electrodes 61, 62 providing an indication of the amount of moisture is transmitted to the control unit 40 which then calculates the amount of moisture, and the arithmetic processing unit 44 calculates the amount of moisture based on the data signal P2 of the amount of moisture.

In this manner, the moisture measurement unit 30′ detects the electrostatic capacity by using the plurality of electrodes 61, 62, and the amount of moisture is measured or determined from the amount of change of the dielectric constant which changes depending on the percentage of moisture content, so that it is possible to measure the amount of moisture in the armpit of the subject according to the electrostatic capacity type. It is possible for the electrostatic capacity to be determined according to the following formula. When assuming that the size S of the sensor surface and the distance d between the electrodes represent constant values, the electrostatic capacity (C) is in proportion to the value of dielectric constant (∈) and the more the amount of moisture is, the larger the values of the dielectric constant and electrostatic capacity become.

Electrostatic capacity(C)=∈×S/d(F)

Dielectric constant=∈
S=size of sensor surface
d=distance between electrodes

Thus, the arithmetic processing unit 44 shown in FIG. 11 forecast-calculates the amount of moisture and the body-temperature of the subject based on the time changes of the data of the amount of moisture and the body-temperature data of the subject, which are obtained from the data P1 of the amount of moisture measured by the moisture measurement unit 30′ and the body-temperature data P2 measured by the temperature measuring unit 31. Therefore, in case of moisture measurement utilizing the electrostatic capacity, it is sufficient if there are provided only two electrodes which are mutually electrically-isolated, and this is relatively easier to implement because it is not necessary to provide a pair of the electrode units for supplying measurement electric-current and a pair of the electrode units for electric-potential measurement respectively such as in a case of the impedance type measurement unit.

The moisture meter disclosed here provides a moisture meter which early-detects the risk of heat illness more accurately by comprehending or identifying two factors which contribute to heat illness, namely the moisture-intake situation of the subject and the outside environment. The moisture meter can be used effectively as an assistance means in which the subject can carry out a proper moisture adjustment. More specifically, the moisture meter sets a Wet-Bulb Globe temperature (WBGT) value from a relationship between the amount of moisture of the subject, which is a moisture-intake situation of the subject, and the temperature and humidity of the environment, which indicate the outside environment, and then the degree of heat-illness risk is judged with reference to a relationship table between the amount of moisture of the subject and the Wet-Bulb Globe temperature (WBGT) value, so that the moisture meter is effective as an assistance means in which it is possible to detect the heat-illness risk early so that it is possible for the subject to carry out proper moisture adjustment.

It is possible for the electric type moisture measurement unit to be one of the impedance type and the electrostatic-capacity type.

In general, it is known that there exist two kinds of sweat glands, apocrine gland and eccrine gland. In case of a human being, the eccrine glands are distributed throughout the whole body, but the apocrine glands exist only in limited portions such as armpits, external auditory canals, lower abdomen, vulvae and the like.

A reason the amount of moisture of the living body of the subject is measured here using the moisture meter and by selecting the armpit as a region of a living body in which the amount of moisture of the subject can be measured properly is because the amount of moisture measured in the armpit is an accurate reflection or indicator of the moisture state of the entire living body.

In general, it is known that when the body-temperature varies, the bioelectrical-impedance value also varies, that is the amount of moisture varies, such that the bioelectrical-impedance value declines when the body-temperature is increased, and the bioelectrical-impedance value is increased when the body-temperature declines. However, according to the moisture meter in the past, the amount of body moisture is calculated from the measured bioelectrical-impedance value without considering anything about the fact that bioelectrical-impedance value varies in this manner caused by the change of the body-temperature, so that it is not possible to find out or determine an accurate amount of body moisture and therefore, it is not possible to detect dehydration accurately.

For example, in a case in which the amount of body moisture is decreased and the body-temperature is increased, the bioelectrical-impedance value increases caused by the decrease of the amount of body moisture, but the bioelectrical-impedance value also declines by virtue of the body-temperature increase, so that even if the judgment is carried out from the amount of body moisture which is calculated from the measured bioelectrical-impedance value, there may occur a situation that the dehydrated state is not detected. For this reason, in case of carrying out the measurement by an impedance method, it is necessary to comprehend the degree of the body-temperature of the measured person, but there has not been carried out a correction for the impedance value according to the measurement of the body-temperature or there has not been carried out an alarm or the like such as a description that an accurate amount of moisture cannot be judged because of developing a fever. That is, as the amount of body moisture decreases, the bioelectrical-impedance value increases. In addition, the body-temperature increases, but the bioelectrical-impedance value declines. So, if the body-temperature is increased and body moisture is decreased, it may not be possible to measure the amount of body moisture by the impedance method to determine the state of dehydration. So, if the measurement is performed with an impedance method, it is necessary to know the body-temperature of the subject.

It is possible for the heat-illness risk display unit 24 of the display unit 20 shown in FIGS. 2A and 2B to display the heat-illness risk-index (degree of heat-illness risk), for example, by three steps of displays, for example “small”, “medium” and “large”. But the moisture meter is not limited in this regard as it is also possible to display the risk of heat-illness by two steps such as “small” and “large”, or by four steps or more.

In the moisture meter mentioned above, the moisture measurement unit 30 of a so-called bioelectrical impedance type (hereinafter, referred to as impedance type) is used, but the moisture meter is not limited in this regard and it is possible to use an optical type moisture measurement unit or a spatial measurement type moisture measurement unit.

It is also possible to employ a constitution in which the sensor unit 27 is attached with a clip or the like and can be hooked on a pocket or the like of a subject's clothing.

The optical type moisture measurement unit can be constructed such that the light-emitting unit illuminates, for example, a light within the infrared region onto the skin of the armpit and the reflected light is light-received by the light-receiving unit. This optical type moisture measurement unit utilizes the phenomenon that the more moisture there is on the skin of the armpit, the more light is absorbed by the moisture content and so the amount of reflected light received by the light-receiving unit is reduced. In the case of a spatial measurement type moisture measurement unit, for example, the vapor of moisture on the skin of the armpit reaches the humidity sensor after passing through a periphery covering member, and the humidity sensor detects the amount of moisture by detecting the humidity inside the space in the inside of the periphery covering member.

Meanwhile, when the moisture meter is positioned at the armpit of the user, the more the full surface of the sensor unit of the distal end is held against the skin, the more accurately it is possible to carry out the measurement. However, it is difficult for the subject or user to visually-observe his/her own armpit, and so it may be difficult for the subject/user to make the sensor unit conform to the correct position.

Furthermore, in case of carrying out the measurement by himself/herself by holding the moisture meter by hand, it is difficult, depending on which portion of the moisture meter the display unit is provided, to refer to the display unit while carrying out the measurement, and it is necessary to confirm the display unit by removing the moisture meter from the armpit.

The following additional embodiment, illustrated in FIG. 13 and representing another example of the body moisture meter disclosed here, addresses these concerns. The body moisture meter 100 shown in FIG. 13 detects the amount of moisture inside the body of the subject by contacting a sensor unit with the skin of the armpit, which is the subject's body-surface and by detecting a physical quantity in response to an electrical signal supplied in the sensor unit. In the body moisture meter 100 relating to this embodiment, the wet condition of the skin of the armpit is detected by measuring the electrostatic capacity of the subject for the aforesaid physical quantity (data relating to the moisture content inside the living body), and the amount of moisture inside the body is calculated. The physical quantity which is detected in order to calculate the amount of body moisture is not limited by the electrostatic capacity and, for example, it is possible to employ an impedance which is measured by supplying a constant electric-voltage or a constant electric-current to the subject.

As shown in FIG. 13, the body moisture meter 100 is provided with a main body unit 110 and an insertion unit 120. The main body unit 110, whose upper surface 114, lower surface 115 and side surfaces 116, 117 are approximately parallel with the longitudinal direction respectively, possesses an overall linear shape. On the housing surface of the main body unit 110, there are arranged various kinds of user interfaces and concurrently, an electronic circuit for calculating the amount of body moisture is housed in the inside of the housing.

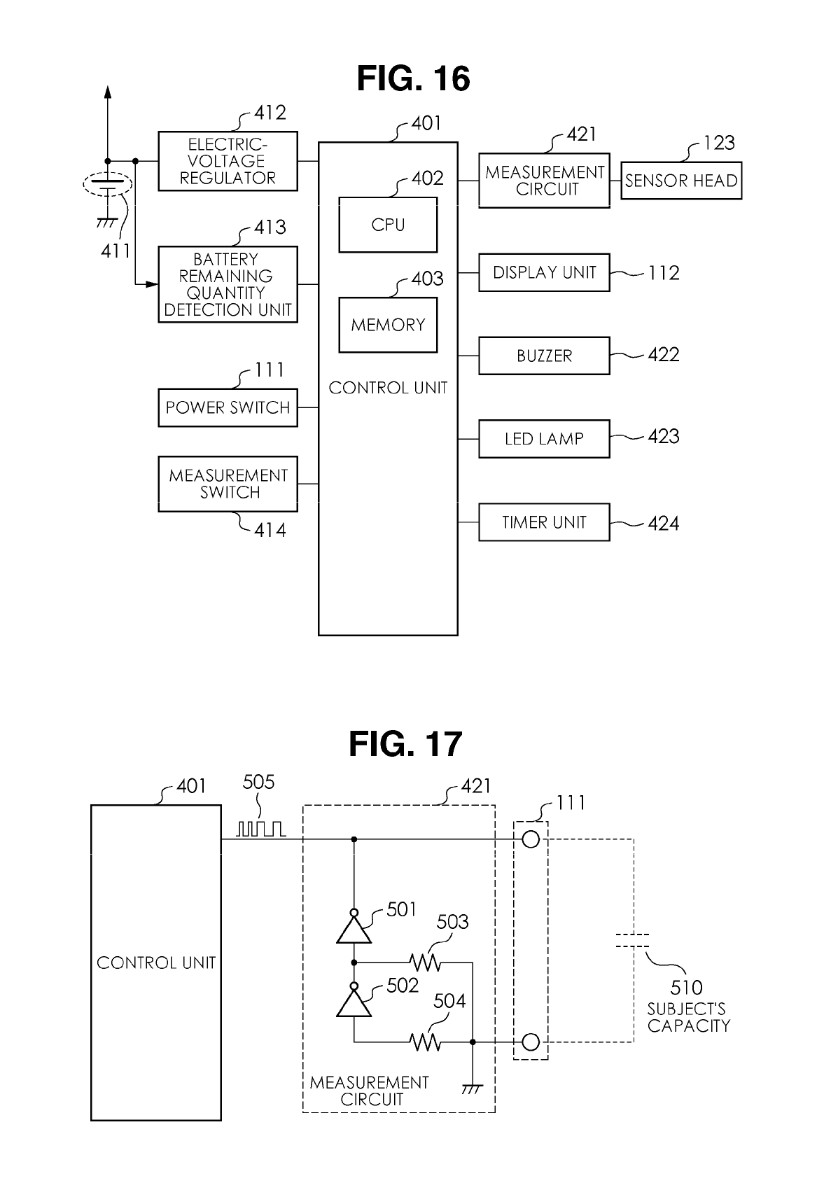

In the example of FIG. 13, there are shown a power switch 111 and a display unit 112 for the user interface. The power switch 111 is disposed at a concave portion at the rear end surface 113 of the main body unit 110. Employing a construction in which the power switch 111 is disposed at a concave portion in this manner makes it possible to prevent miss-operation of the power switch 111. When the power switch 111 is turned on, there is started the power supply to each unit of the body moisture meter 100 from a power unit 411 (FIG. 16) which will be discussed below, and the body moisture meter 100 is in an operational state.

The display unit 112 is disposed slightly more to the front side in the longitudinal direction on the side surface 117 of the main body unit 110. This is because there will not occur a case in which the display unit 112 is covered completely by the grasping hand of the measurer (user) even in a case in which the measurer grasps the grasp area 118 on an occasion of measuring the amount of body moisture of the subject by using the body moisture meter 100 (in order to make it possible to visually-confirm the measurement result even in the grasp state).

The display unit 112 displays a measurement result 131 of the amount of moisture for a current measurement. The display unit 112 is an example of display means for displaying the amount of body moisture. In addition, there is also displayed the previous measurement result (“last time”) 132 concurrently as a reference. Further, on a battery display unit 133, there is displayed the remaining quantity of the battery (power unit 411 of FIG. 16). Also, in a case in which the invalid measurement result is obtained or in a case in which the measurement error is detected, there is displayed “E” on the display unit 112 and that effect is reported to the user. It is assumed that the character or the like, which is displayed on the display unit 112, is to be displayed such that the upper surface 114 side of the main body unit 110 is “up” and the lower surface 115 side is “down”. That is, the characters displayed at the display unit 112 are displayed to be properly read when the upper surface 114 of the main body unit 110 is up and the lower surface 115 is “down”.

The insertion unit 120 of the body moisture meter 100, whose upper surface 124 and lower surface 125 have curved shapes (i.e., the upper surface 124 and the lower surface 125 are both curved), is curved gently downward as a whole with respect to the main body unit 110. A sensor unit 121 is slidably held on or mounted at the distal surface 122 of the insertion unit 120.

The sensor unit 121 includes a sensor head 123 having a surface approximately parallel with the distal surface 122 and is biased toward an arrow 141b direction by a spring (for example, by a biasing force of around 150 gf) in order to secure the depression under a condition for assuring a close contact of the sensor head 123 with the skin. Then, when the sensor head 123 is pressed onto the skin of the armpit of the subject, the sensor unit 121 slides in an arrow 141a direction (direction approximately perpendicular to the distal surface 122, that is, normal-line direction of the distal surface 122) as much as a predetermined amount (for example, 1 mm to 10 mm, and 4 mm in this embodiment disclosed by way of example), and by this operation the measurement will start (hereinafter, arrow 141a direction is referred to as slide direction).

Specifically, after the user turns on the power switch 111 and the body moisture meter 100 is set as an operation state and when it is detected that the sensor head 123 is pressed onto the armpit of the subject for a predetermined time or more (for example, for two seconds or more), the measurement of the amount of body moisture will be started. Alternatively, after the user turns on the power switch 111 and the body moisture meter 100 is set as an operation state and when it is detected that the sensor head is pressed onto the armpit of the subject with a predetermined load (a load of, for example, 20 gf to 200 gf, more preferably, 100 gf to 190 gf, and 150 gf in this embodiment disclosed by way of example), the measurement amount of body moisture will be started. Depending on such a mechanism, it is possible, for the degree of close contact of the sensor head 123 with the armpit at the time of measurement to be made constant.

On the contact surface between the sensor head 123 and the subject, electrodes are laid-down and there is provided a protection member to cover the electrodes. The contact surface of the sensor head 123 is not limited by the flat-surface shape, and it is possible to employ a convexly curved shape. An example of such a shape of the contact surface is a shape which is formed as a portion of a spherical surface (for example, spherical surface having radius of 15 mm).

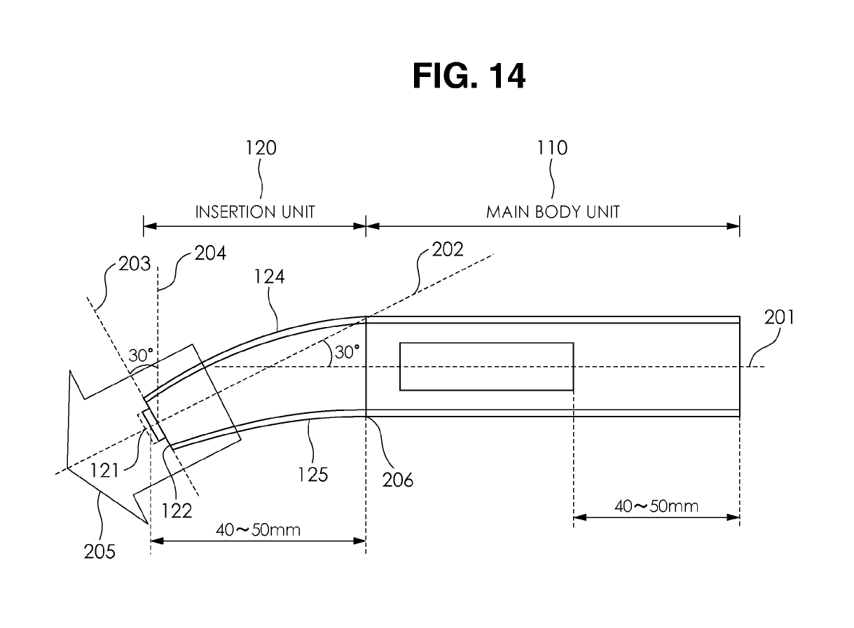

Next, there will be explained a housing shape of the body moisture meter 100 in detail. FIG. 14 is a view for explaining the housing shape of the body moisture meter 100 in detail.

As shown in FIG. 14, with regard to the insertion unit 120 of the body moisture meter 100, the distal surface 122 is formed such that a normal-line or perpendicular 202 (in other words, slide direction) of the distal surface 122 forms an angle of approximately 30° with respect to a longitudinal direction or longitudinal central axis 201 of the main body unit 110. Stated differently, the distal surface 122 is formed such that a direction 203 parallel to the distal surface 122 forms an angle of approximately 30° with respect to a direction 204 perpendicular to the longitudinal direction or longitudinal central axis 201 of the main body unit 110. In addition, the housing in the vicinity of the distal surface 122 of the insertion unit 120 has a shape which is roughly along the normal-line direction 202 of the distal surface 122. That is, the insertion unit is bent relative to the main body unit and so the approach to the armpit is relatively simple.

Because the curved shape of the insertion unit 120 is formed to coincide with the curved-surface direction 205 of the insertion unit 120 and the slide direction 202 of the sensor unit 121, it is possible for the measurer (user), on an occasion when the measurer grasps the body moisture meter 100 and presses it onto the armpit of the subject at the time of measurement, to carry out the measurement only by depressing the body moisture meter 100 toward the curved-surface direction 205 without making a mistake about the depression direction even in a state of not being able to visually-confirm the distal surface 122. In other words, it is possible to make the sensor unit 121 closely in contact with the armpit of the subject precisely and it becomes possible to realize an accurate measurement.

Also, as shown in FIG. 14, with regard to the insertion unit 120 of the body moisture meter 100, the lower surface 125 of the insertion unit 120 has a curved shape. In this manner, by forming the lower surface 125 of the insertion unit 120 in a curved shape, it becomes possible, on an occasion when the measurer grasps the body moisture meter 100 and presses it onto the armpit of the subject at the time of measurement, to avoid the side wall of the front-side of the upper arm (inner surface of the upper arm) of the subject and the lower surface 125 of the body moisture meter 100 from interfering even in a case in which the armpit of the subject is deep.

Further, as shown in FIG. 14, with regard to the insertion unit 120 of the body moisture meter 100, the length of the insertion unit 120 is defined such that the sensor unit 121 is located at a position spaced apart by as much as approximately 40 mm to 50 mm from the boundary position 206 between the main body unit 110 and the insertion unit 120. That is, the length of the insertion unit, measured as the horizontal distance from the boundary to the center distal-most point of the sensor unit while the main body unit is horizontally positioned is 40 mm to 50 mm. The boundary position 206 represents the place at which the straight main body unit 110 transitions to or is connected to the curved insertion unit 120.

By defining the length of the insertion unit 120 in this manner, even in a case in which the armpit of the subject is deep, it is possible for the measurer to press the sensor unit 121 onto the armpit of the subject without a phenomenon that the grasping hand interferes with the upper arm or the like of the subject.

Further, as shown in FIG. 14, the insertion unit 120 is formed such that the cross-sectional area of the insertion unit 120 is equal to the cross-sectional area of the main body unit 110 at the boundary position 206 and is formed so as to become smaller gradually along the approach to the sensor unit 121. That is, the insertion unit 120 is formed so as to become slimmer toward the distal end.

In this manner, by reducing the cross-sectional area in the vicinity of the sensor unit 121 of the insertion unit 120, it is possible, on an occasion when the measurer inserts the body moisture meter 100 into the armpit of the subject, to carry out the insertion relatively easily even in a case of a subject who has a narrow variable range for his upper arm.

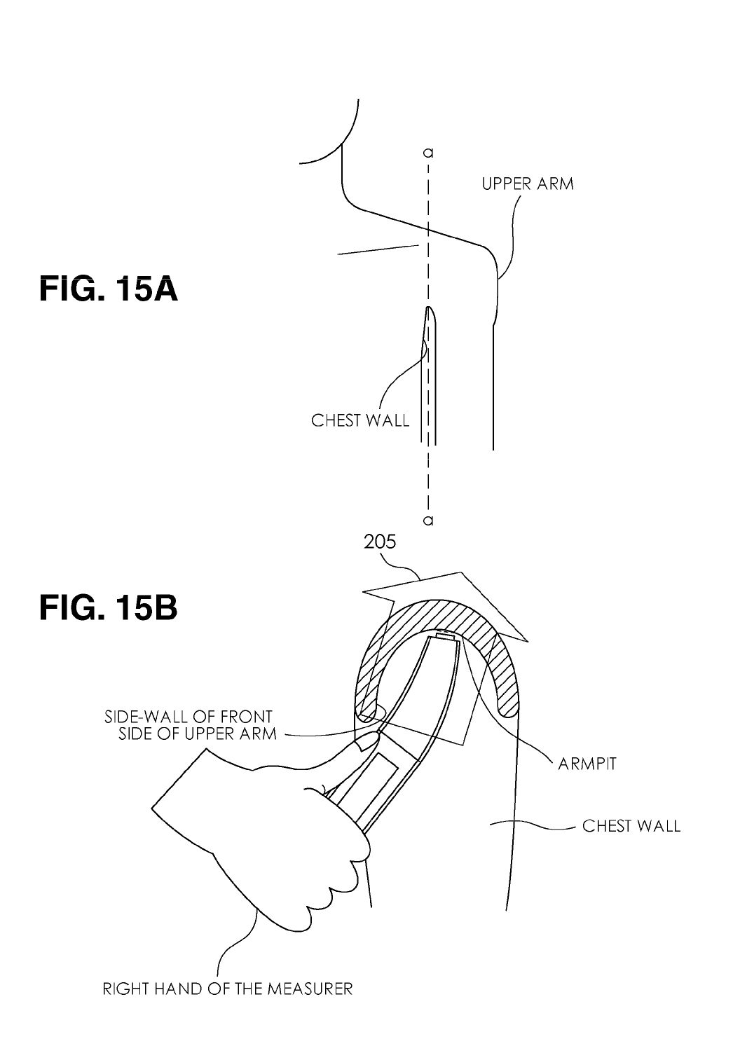

Set forth next, with reference to FIGS. 15A and 15B, is a description of an example of a manner of using the body moisture meter 100 having the aforesaid unique outward-appearance shape. FIG. 15A shows the left upper half of the body of the measured person and FIG. 15B shows the cross-section at the section line 15B-15B in FIG. 15A.

As shown in FIG. 15B, the body moisture meter 100 carries out the measurement of the amount of body moisture of the subject in a state in which the sensor unit 121 is pressed onto the armpit between the left upper arm and the left chest wall of the subject.

On an occasion of pressing the sensor unit 121 onto the armpit, the measurer grasps the grasp area 118 of the body moisture meter 100 by the right hand such that the sensor unit 121 faces to the upper side, and inserts the sensor unit 121 toward the armpit from the front lower side of the subject.

As mentioned above, the insertion unit 120 of the body moisture meter 100 is curved gently and also the length from the boundary position 206 to the sensor unit 121 possesses a length of around 40 mm to 50 mm, so that when this insertion unit 120 is inserted from the front lower side of the subject toward the armpit, it is possible to press the sensor unit 121 onto the armpit approximately perpendicularly without the side wall of the front side of the upper arm (inner surface of the upper arm) and the body moisture meter 100 interfering with each other and also, without the right hand of the measurer interfering with the upper arm of the subject.

Also, the curved shape of the insertion unit 120 is formed such that the curved-surface direction 205 of the insertion unit 120 and the slide direction 202 of the sensor unit 121 coincide with each other, so that it is possible for measurer to press the sensor unit 121 onto the armpit approximately perpendicularly by pressing it along the curved-surface direction 205.

In this manner, by virtue of the shape of the body moisture meter 100 according to his embodiment disclosed by way of example, it is possible to carry out the measurement rather easily even in a case of a subject who has a deep armpit.

FIG. 16 is a block diagram showing an example of the construction of the body moisture meter 100 according to the embodiment shown in FIGS. 13, 14, 15A and 15B. In FIG. 16, the control unit 401 includes a CPU 402 and a memory 403, and the CPU 402 executes various controls in the body moisture meter 100 by executing programs stored in the memory 403.

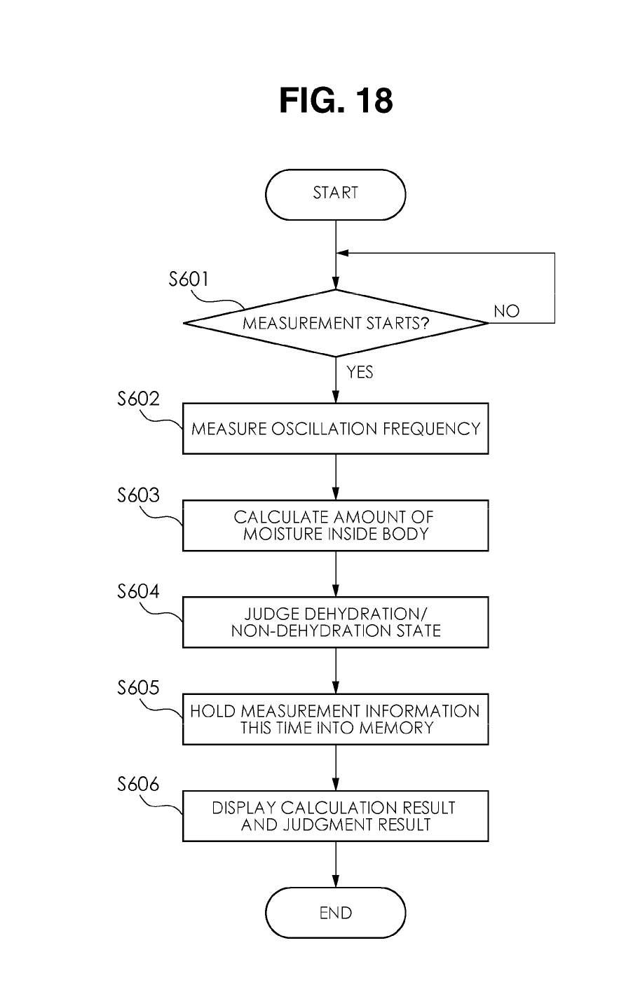

For example, the CPU 402 executes a display control of the display unit 112 which will be described later by a flowchart shown in FIG. 18, drive controls of a buzzer 422 and an LED lamp 423, the measurement of the amount of body moisture (electrostatic-capacity measurement in this exemplified embodiment) and the like. The memory 403 includes a nonvolatile memory and a volatile memory, with the nonvolatile memory being utilized as a program memory and the volatile memory being utilized as a working memory of the CPU 402.

The power unit 411 includes an exchangeable battery or a rechargeable battery and supplies electric-power to the respective units of the body moisture meter 100. A voltage regulator 412 supplies a constant electric-voltage (for example, 2.3V) to the control unit 401 and the like. A battery remaining-quantity detection unit 413 detects the remaining quantity of the battery based on a voltage value supplied from the power unit 411 and provides notification of the detection result to the control unit 401. The control unit 401 controls the display of the battery display unit 133 based on the battery remaining-quantity detection signal from the battery remaining-quantity detection unit 413.

When the power switch 111 is depressed, the power supply from the power unit 411 to the respective units is started. Then, when detecting that the depression of the power switch 111 by the user is continued for one second or more, the control unit 401 maintains the power supply from the power unit 411 to the respective units and sets the body moisture meter 100 to be in an operation state. As mentioned above, a measurement switch 414 becomes in an ON-state when the sensor unit 121 is pressed by a predetermined amount or more in the arrow 141a direction. The control unit 401 starts the measurement of the amount of moisture when the ON-state of the measurement switch 414 is continued for a predetermined time period (for example, for two seconds). To prevent the consumption of power in the power unit 411, in a case in which the measurement will not start even if five minutes elapse after the body moisture meter 100 becomes in an operation state, the control unit 401 makes the body moisture meter 100 shift to a power OFF state automatically.

A measurement circuit 421 is connected with the sensor head 123 and measures the electrostatic capacity. FIG. 17 is a diagram showing an example of the configuration of the measurement circuit 421. There is formed a CR oscillation circuit by operational amplifiers 501, 502; resistors 503, 504; and a subject's capacity 510. The oscillation frequency of the output signal 505 changes in response to the subject's capacity 510, so that the control unit 401 calculates the subject's capacity 510 by measuring the frequency of the output signal 505. It is assumed that the sensor head 123 of this embodiment has a construction in which, for example, two comb type electrodes are disposed such that the respective comb teeth are aligned alternately. But the invention here is not limited by this configuration.

Referring once again to FIG. 16, the display unit 112 carries out such a display as shown in FIG. 13 under the control of the control unit 401. The buzzer 422 sounds when the measurement is started by the depression of the sensor unit 121 and when the measurement of the amount of body moisture is completed, in which the start and the completion of the measurement are notified to the user. The LED lamp 423 carries out a similar notice as that of the buzzer 422. More specifically, the LED lamp 423 is turned on when the measurement is started by the depression of the sensor unit 121 and when the measurement of the amount of body moisture is completed, in which the start and the completion of the measurement are notified to the user. The timer unit 424 operates by receiving the power supply from the power unit 411, even if the power is in an OFF-state, and notifies the clock time to the control unit 401 during the operation state.

Referring to FIG. 18, there will be explained an example of an operation of the body moisture meter 100 having the construction as described above.

In step S601, the control unit 401 detects an instruction of the measurement-start. In this embodiment, the state of the measurement switch 414 is monitored and in a case in which the ON-state of the measurement switch 414 is continued for two seconds or more, it is judged that the instruction of the measurement-start was detected. When detecting the instruction of the measurement-start, the control unit 401 measures, in step S602, the oscillation frequency of the output signal 505 from the measurement circuit 421.

In step S603, the amount of body moisture of the subject is calculated based on the oscillation frequency of the output signal 505 measured in step S602.

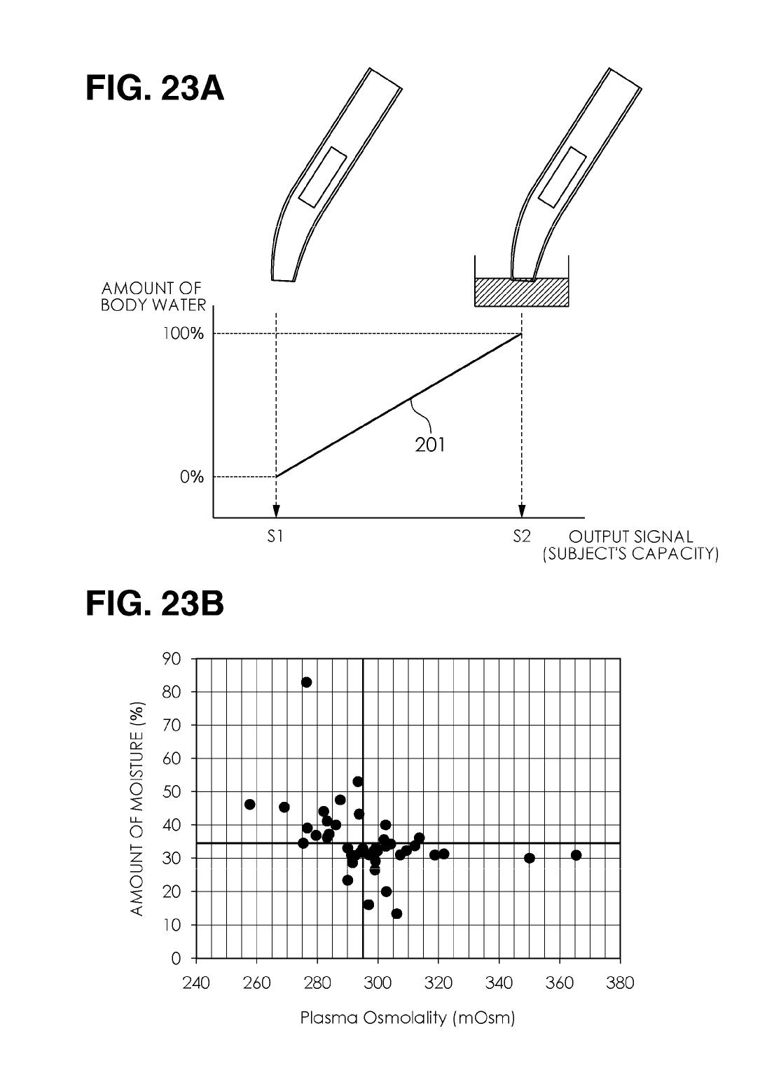

In step S604, it is judged whether or not the subject is in a dehydrated state based on whether or not the amount of body moisture calculated in step S603 exceeds a predetermined threshold value. It is preferable for the threshold value in this case to be, for example, a value corresponding to 35% considered in the context of water representing a value of 100% and the air representing a value of 0%. These references to water representing a value of 100% and air representing a value of 0% means, for example, as indicated in FIG. 23A, when measured in the state that the sensor unit of the body moisture meter is soaked in water within a glass, the measured value indicates 100%, and in a state in which the sensor unit contacts nothing, the measured value indicates 0%.

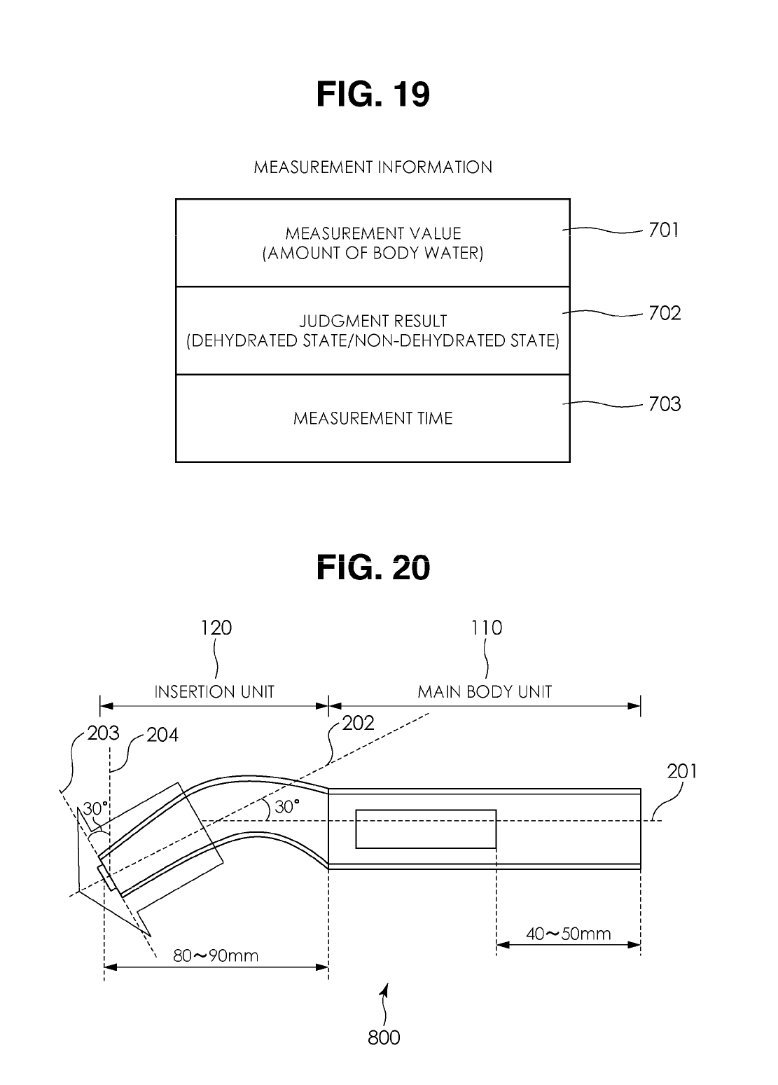

In step S605, the measurement information this time is stored in the memory 403. FIG. 19 is a diagram showing a data structure of the measurement information which is stored in the memory 403. In FIG. 19, a measurement value 701 is the amount of body moisture calculated by the measurement this time. A judgment result 702 is the information indicating a dehydrated state or a non-dehydrated state, which was judged in step S604 with respect to the amount of body moisture calculated by the measurement this time. A measurement time 703 is the information indicating the time notified from the timer unit 424 in the measurement this time. It is possible for measurement time 703 to be set, for example, as the time instant which was notified from the timer unit 424 at the point in time when the measurement is carried out in step S602. The measurement time 703 is information that records the time of measurement, and the timer unit 424 measures the time.

In step S606, the amount of body moisture calculated by the measurement this time is displayed on the display unit 112. At that time, the display is carried out by the display mode in response to the judgment result of the dehydrated state or the non-dehydrated state. For example, in case of the dehydrated state, the amount of body moisture is displayed by a red color, and in case of the non-dehydrated state, the amount of body moisture is displayed by a blue color.

As clear from the explanation above, with regard to the body moisture meter 100 relating to this embodiment, in order for the armpit to be a place which is suitable for the measurement region, there is employed a constitution in which the distal surface is formed such that the normal-line direction of the distal surface forms an angle of approximately 30° with respect to the longitudinal direction of the main body unit.

The distal end of the insertion unit is formed or configured to possess a shape which is along the normal-line direction of the distal surface.

The lower surface side of the insertion unit possesses a curved shape.

The length of the insertion unit is defined such that the distance between the sensor unit and the boundary position is 40 mm to 50 mm.

The insertion unit is configured so that it becomes slimmer toward the distal end.

The body moisture meter is thus configured in a way allowing the armpit to be used as the measurement region, to provide a structure by which the measurement is relatively easy.

The above-described fourth embodiment possesses a shape such that the insertion unit 120 is curved toward the downward-direction from the boundary position 206 (that is, a shape in which the upper surface 124 of the insertion unit 120 is positioned downward from the upper surface 114 of the main body unit 110), but the invention here is not limited by this configuration. For example, it is possible to employ a shape such as shown in FOG. 20 in which a portion of the upper surface 124 of the insertion unit 120 is positioned upward from the upper surface 114 of the main body unit 110.

FIG. 20 illustrates an outward-appearance configuration of a body moisture meter 800 according to a fifth embodiment representing another example of the moisture meter disclosed here. The insertion unit 120 having a shape as shown in FIG. 20 is able to obtain a similar effect as that of the aforesaid fourth exemplified embodiment.

In this embodiment of the moisture meter, the upper surface of the insertion unit is curved and configured so that with the moisture meter in the horizontal position shown in FIG. 20, the upper curved surface of the insertion unit 120, at a position forward/distal of the boundary between the insertion unit 120 and the main body unit 110, extends above a horizontal plane containing the upper surface of the main body unit 110. Stated differently, in the view shown in FIG. 20 in which the moisture meter is horizontally positioned, an imaginary continuation of the top surface of the main body unit passes through or intersects a part of the insertion unit 120 at a position forward/distal of the boundary between the insertion unit 120 and the main body unit 110. The bottom surface of the insertion unit 120 is curved and configured so that with the moisture meter in the horizontal position shown in FIG. 20, the lower curved surface of the insertion unit 120, at a position forward/distal of the boundary between the insertion unit 120 and the main body unit 110, extends above a horizontal plane at the lower surface of the main body unit 110. Stated differently, in the view shown in FIG. 20 in which the moisture meter is horizontally positioned, an imaginary continuation of the bottom surface of the main body unit passes through or intersects a part of the insertion unit 120 at a position forward/distal of the boundary between the insertion unit 120 and the main body unit 110.

The description above about the fourth embodiment did not specifically describe the gravity-center position of the body moisture meter 100. It is possible, though not always necessary, for the gravity-center position of the body moisture meter 100 to be at the center position of the main body unit 110.

As described above, the measurer grasps the body moisture meter 100 by directing the sensor unit 121 upward at the time of measurement, so that by disposing, for example, the power unit 411 and the control unit 401, on the rear end surface 113 side of the main body unit 110, the center of gravity of the moisture meter shifts to the rear end surface 113 side of the main body unit 110 and it becomes relatively easy for the measurer to stabilize the center of gravity and obtain the proper or desired balance at the time of measurement.

Also, by virtue of the fact that the body moisture meter 100 is grasped by setting the upper surface 114 side in a downwardly facing direction at the time of measurement, it is easier for the measurer to stabilize the center of gravity and obtain the proper or desired balance at the time of measurement by disposing the gravity center on the upper surface 114 side (side opposite to the curved-surface direction of the insertion unit 120) of the main body unit 110.

In the above-described embodiments, the moisture meter is configured so that a line normal (perpendicular) to the plane of the distal surface 122 forms an angle of approximately 30° with respect to the longitudinal direction 201 of the main body unit 110. But the invention here is not limited in this regard. For example, it is possible for the distal surface 122 to be formed such that a line normal to the plane of the distal surface 122 forms an angle of approximately 20° to 40° with respect to the longitudinal direction 201 of the main body unit 110.

Also, in the fourth embodiment described above, the length of the insertion unit 120 is defined such that the distance from the sensor unit 121 to the boundary position 206 is around 40 mm to 50 mm. But the invention here is not limited in this regard. For example, in consideration of the depth of the armpit of the subject, it is possible for the length of the insertion unit 120 to be defined such that the distance from the sensor unit 121 to the boundary position 206 is around 80 mm to 90 mm.

Also, in the above-described sixth embodiment, the distance from the rear end surface 113 to the display unit 112 is around 40 mm to 50 mm, but the invention here is not limited in this regard. The display unit 112 can be disposed in a different range so that the display unit 112 will not be covered completely when the measurer grasps the main body unit 110.

The amount of moisture of the armpit is a property in which there is maintained a stabilized state specified by the person, similar to a “normal body-temperature” for the body-temperature. That is, individuals typically have a certain amount of moisture as measured at the armpit, but it is unusual and difficult for each person to memorize the amount of moisture representing the person's “normal” moisture amount (“stabilized amount of moisture” corresponding to the “normal body-temperature”), which is maintained stably. Also, with regard to such a judgment of whether such a stable amount of moisture of the individual person is rather high or rather low, it is not possible to judge if the there is no specific target.

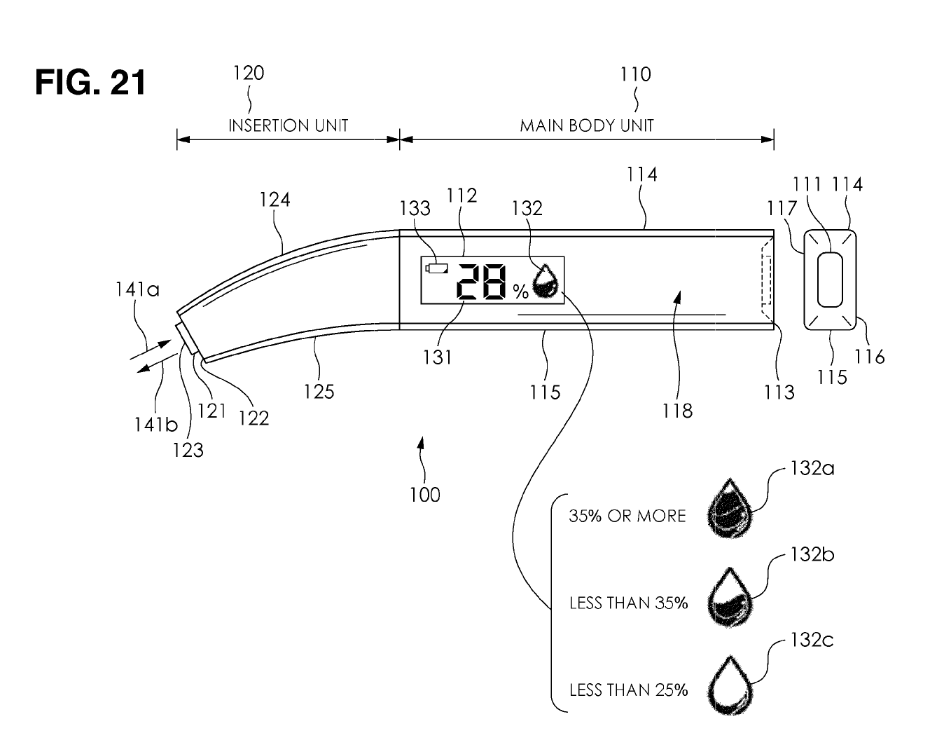

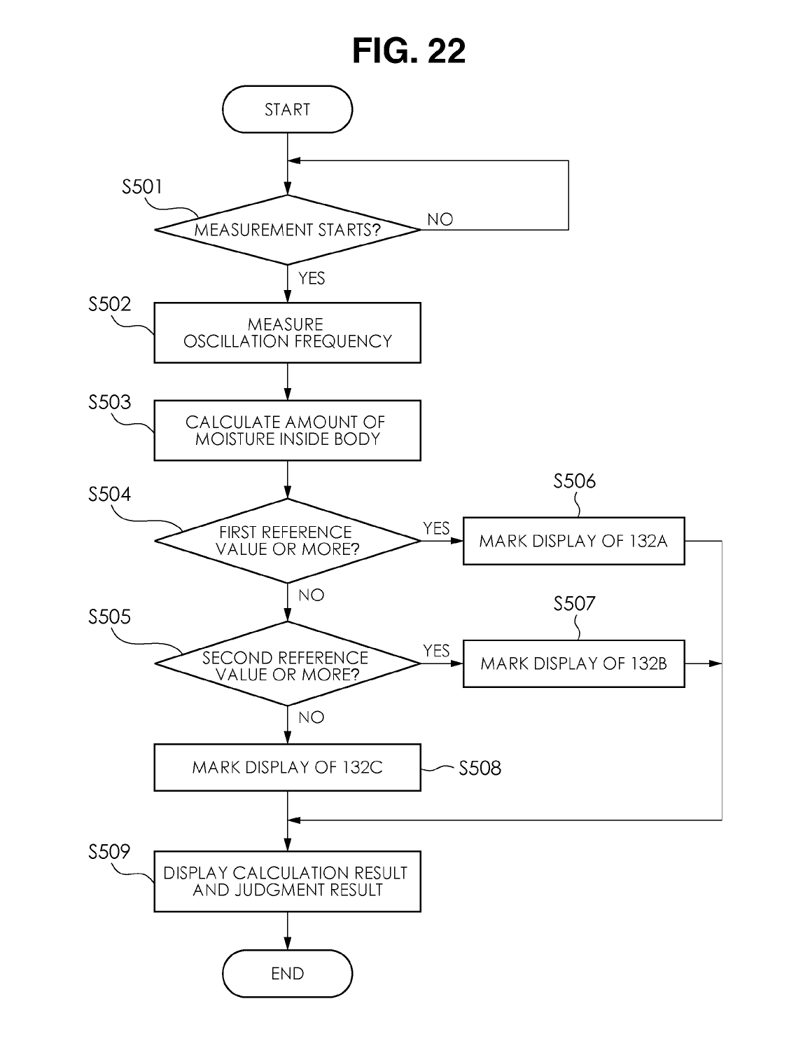

The following eighth embodiment of the moisture meter shown in FIGS. 21 and 22 addresses the concern expressed above.

The external shape of the body moisture meter and many of features of the eighth embodiment, including the electrical constitution, are similar to those of each of the fourth to seventh embodiments, and the features which are the same as the earlier embodiments are identified by common reference numerals and a detailed description of such features is not repeated.

Referring to FIG. 21, the display unit 112 displays the measurement result 131 of the amount of moisture. The display unit 112 also displays the degree of possibility of dehydration and a mark 132 which indicates the degree of seriousness of the risk of dehydration as a reference item. In this embodiment disclosed as an example, there are respectively displayed: a mark 132a illustrating a relatively completely filled-in water-drop when the measurement result of the amount of moisture is 35% or more, which indicates the amount of moisture is in a normal state; a mark 132b illustrating a half-filled water-drop when the measurement result of the amount of moisture is less than 35% and 25% or more indicating the amount of moisture in the body is slightly insufficient and also that there is a possibility of dehydration; and a mark 132c illustrating an empty-water-drop when the measurement result of the amount of moisture is less than 25% indicating the body is in a dehydrated state and also that there is a possibility of being in a serious condition.

The battery display unit 133 displays the remaining quantity of the battery (power unit 411 of FIG. 16). Also, in a case in which an invalid measurement result is obtained and a measurement error is detected, “E” is displayed on the display unit 112 and that effect is informed to the user. There will be employed a configuration in which the characters or the like which are displayed on the display unit 112 are displayed by setting the upper surface 114 side of the main body unit 110 as an upside and the lower surface 115 side as a down side.

Set forth next is a description of an example of usage of the body moisture meter 100 having the unique outward-appearance shape. FIGS. 15A and 15B are views explaining an example of using the body moisture meter 100, in which FIG. 15A shows the left upper half of the body of the measured person and FIG. 15B shows a cross-section at the section line 15B-15B in FIG. 15A. As shown in FIG. 15B, the body moisture meter 100 carries out the measurement of the amount of body moisture (body moisture) of the subject in a state in which the sensor unit 121 is pressed onto the armpit between the left upper arm and the left chest wall of the subject.

On an occasion of pressing the sensor unit 121 onto the armpit, the measurer grasps the grasp area 118 of the body moisture meter 100 by the right hand such that the sensor unit 121 is directed to the upper side and the sensor unit 121 is inserted from the front lower side of the subject toward the armpit.

As shown in FIG. 21, the insertion unit 120 of the body moisture meter 100 is curved gently and when this is inserted from the front lower side of the subject toward the armpit, it is possible to press the sensor unit 121 onto the armpit approximately perpendicularly without the side wall of the front side of the upper arm (inner surface of the upper arm) and the body moisture meter 100 interfering with each other and also, without a phenomenon that the right hand of the measurer interferes with the upper arm of the subject.

Also, the curved shape of the insertion unit 120 is formed such that the curved-surface direction of the insertion unit 120 and the slide direction 141 of the sensor unit 121 coincide with each other, so that it is possible for measurer to press the sensor unit 121 onto the armpit approximately perpendicularly by pressing it along the curved-surface direction 205.

In this manner, according to the shape of the body moisture meter 100 relating to this embodiment, it is possible to carrying out the measurement easily even in a case of a subject who has a deep armpit.

There will next be explained the operation of the body moisture meter 100 according to this embodiment, which is constructed as described above, with reference to the flowchart of FIG. 22.

In step S501, the control unit 401 detects an instruction of the measurement-start. That is, the control unit 401 determines that the user wishes to start a moisture measurement using the moisture meter. In this embodiment, this is accomplished by monitoring the state of the measurement switch 414, and when it is determined that an ON-state of the measurement switch 414 is continued for two seconds or more, it is judged that an instruction of the measurement-start is detected (i.e., that the user wishes to perform a moisture measurement). When the control unit 401 detects the instruction of the measurement-start, in step S502, the control unit 401 measures the oscillation frequency of the output signal 505 from the measurement circuit 421. In step S503, the control unit 401 calculates the amount of body moisture (body moisture) of the subject based on the oscillation frequency of the output signal 505 which was measured in step S502.

In step S504, the control unit 401 judges whether the amount of body moisture (body moisture) calculated in step S503 is equal to or greater than a first reference value (35% in this embodiment). If it is determined in step S504 that the calculated amount of body moisture is not equal to or greater than the first reference value (NO in step S540), it is determined in step S505 whether the calculated amount of body moisture is equal to or greater than a second reference value (25% in this embodiment). In a case in which the amount of body moisture is equal to or greater than the first reference value, the process proceeds to step S506 and the control unit 401 selects the mark 132a which indicates that the body moisture value is a normal value without fear or risk of dehydration. In a case in which the amount of body moisture is less than the first reference value but is equal to or greater than the second reference value (i.e., NO in step S504 and YES in step S505), the process proceeds to step S507 and the control unit 401 selects the mark 132b which indicates that there is a possibility of dehydration. Further, in a case in which the amount of body moisture is less than the second reference value (i.e., NO in step S505), the process proceeds to step S508 and the control unit 401 selects the mark 132c which indicates that the body is in a dehydrated state. The marks 132a-132c represent an example of changing means for changing the display mode by the display unit to call the users' attention to a situation in which the amount of body moisture is lower than a reference value. In this embodiment, the display modes are changed in response to the first reference value and the second reference value (35% and 25%), but the invention is not limited in this aspect. For example, it is possible to change the display modes only in response to the first reference value and it is possible to change the display modes sequentially in response to three reference values or more.

Next, in step S509, the control unit 401 displays the amount of body moisture, which was calculated by the measurement this time, as a measurement result 131 on the display unit 112. At that time, the control unit 401 also displays the mark 132, which was selected by any one of the aforesaid steps S506 to S508, on the display unit 112. It is possible for the user to comprehend the measurement value of the amount of body water and concurrently, to judge whether it is in a dehydrated state or in a non-dehydrated state by the display of the mark 132 and to judge the degree of seriousness thereof rather easily.

Next, there will be explained a calibration method of the body moisture meter according to this embodiment, and there will be explained the first reference value and the second reference value, which were mentioned above. As shown in FIG. 23A, in this embodiment, in a case in which the output signal 505 (subject's electrostatic-capacity) when carrying out the measurement in the air using the body moisture meter 100 is S1 and the output signal 505 (subject's electrostatic-capacity) when carrying out the measurement in water is S2, a 0% amount of body moisture is allotted to the S1 and a 100% amount of body moisture is allotted to the S2. Then, by using a straight line 201 in which the amount of body moisture is allotted linearly to the output signal between the S1 and the S2, the output signal from the sensor is stored in the nonvolatile memory of the memory 403 by determining parameters such that the output signal is to be converted to the amount of body moisture. The moisture measurement unit 30 under the control of the control unit 40 is an example of conversion means for converting the signal from the sensor unit to the amount of body moisture. In step S503, by using parameters stored in the nonvolatile memory, the subject's electrostatic-capacity is converted to the amount of body moisture. That is, the FIG. 23A graph provides a correlation between the subject's electrostatic-capacity and the amount of body moisture so that after a subject's electrostatic-capacity is measured, the measurement result of the subject's electrostatic-capacity can be applied to the FIG. 23A graph to determine the subject's amount of body moisture.

In FIG. 23B, there is shown a result which was obtained by measuring the amounts of body moisture in the armpits with respect to a plurality of subjects using the body moisture meter 100 for which such a calibration was carried out and concurrently, which was obtained by measuring plasma osmolalities depending on blood tests. In general, it is judged that the subject whose plasma osmolality is 295 mmOsm or more is in a dehydrated state. The measurement results in the drawing show it was possible to obtain such a result in which the measurement results for the amounts of body moisture according to the body moisture meter 100 were 35% or less with respect to 85% or more of the subjects within the subjects whose plasma osmolalities were 295 mmOsm or more. In other words using the moisture meter 100 on all of the subjects whose plasma osmolalities were 295 mmOsm, the moisture meter 100 determined that 85% of the subjects had a body moisture amount of 35% or less. Also, with respect to approximately 100% of the subjects whose plasma osmolalities are 295 mmOsm or more, the measurement results of the amounts of body moisture according to the body moisture meter 100 are 40% or less and with respect to approximately 100% of the subjects whose plasma osmolalities are 295 mmOsm or less, the measurement results of the amounts of body water according to the body moisture meter 100 are 25% or more. Consequently, it is conceivable that a value between 25% and 40% can be set for the first reference value, but it is considered preferable for a general target to use 35% for the first reference value of the amount of body moisture, which can be applied to 85% or more of the subjects. Note that 25% is to be used with regard to the second reference value.

As clear from the explanation above, according to the body moisture meter 100 according to this embodiment, it is possible for the user, from the display mode of the mark 132, to judge whether or not the user is in a dehydrated state and also to judge the degree of seriousness of the dehydrated state in a manner like a body-temperature measurement.

In the embodiment described above, the first reference value and the second reference value are fixed values, but the invention is not limited in this regard. For example, it is possible to employ a configuration in which the user can set the first reference value within a range of 25% to 40%, which was discussed above. In this case, it is possible to employ a configuration in which the second reference value can be set separately within a range lower than that of the first reference value and it is also possible to employ a configuration in which a value obtained by subtracting a predetermined value from the first reference value is set automatically. If employing such a configuration, it is possible to cancel the personal difference which appears in the measurement value of the amount of body moisture at normal time.

Also, in the aforesaid embodiments, the changes of the display modes, in a case in which the measurement result becomes lower than the first reference value or becomes lower than the second reference value, are carried out by the changes of the waterdrop marks, but the invention is not limited in this regard. It is sufficient if the display modes are to be changed, for example, by changing the display colors or the like, such that it is possible to notify the user about the fact that the value has become lower than the reference value and this notification is brought to the user's attention.

Also, the first reference value and the second reference value which are defined in this embodiment must be understood as the values corresponding to predetermined values (35% and 25% in the embodiment) in a case in which the signals outputted when the water is measured and when the air is measured are allotted to 100% of and 0% of the amount of moisture respectively, and the signal outputted from the sensor unit 121 and the amount of moisture are correlated to each other in a linear relation. In this embodiment, the calibration method of the sensor unit 121 and the definition of the reference value are in conformity with each other, and this is because it is possible for the first reference value and the second reference value to be different values from 35% and 25% if the calibration method of the sensor unit 121 is different.

The detailed description above describes features and aspects of embodiments of a moisture meter representing examples of the moisture meter disclosed here. The present invention is not limited, however, to the precise embodiments and variations described. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. A moisture meter measuring moisture content of a subject comprising:
a moisture measurement unit configured to be positioned in contact with skin surface of an armpit of a subject and held by the armpit of the subject to measure an amount of moisture of the subject through the contact with the skin surface of the armpit;
a sensor unit which measures temperature of an environment in which the subject is located as well as humidity of the environment; and
a processing unit which obtains the amount of moisture of the subject measured by the moisture measurement unit, which sets a Wet-Bulb Globe temperature (WBGT) value using a relationship between the temperature and the humidity measured by the sensor unit, and which determines a heat illness risk-index by referencing a relationship table setting forth a relationship between the amount of moisture of the subject and the Wet-Bulb Globe temperature (WBGT) value.

2. The moisture meter according to claim 1, further comprising:
a main body unit possessing one end and an other end opposite the one end;
a measurement-unit holding unit disposed at one end of the main body unit and configured to be positioned in the subject's armpit while sandwiched between an arm of the subject and a facing body part of the subject, the moisture measurement unit being located in the main body unit;
a display-unit holding unit disposed at the other end of the main body unit, with a display unit provided at the display-unit holding unit to display the amount of moisture of the subject measured by the moisture measurement unit and the heat-illness risk-index; and
electrical wiring connecting the sensor unit to the other end of the main body unit so that the sensor unit is movable relative to the main body unit.

3. The moisture meter according to claim 1, further comprising:
a main body unit possessing one end and an other end opposite the one end;
a measurement-unit holding unit disposed at one end of the main body unit and configured to be positioned in the subject's armpit while sandwiched between an arm of the subject and a facing body part of the subject, the moisture measurement unit being located in the main body unit;
a display-unit holding unit disposed at the other end of the main body unit, with a display unit provided at the display-unit holding unit to display the amount of moisture of the subject measured by the moisture measurement unit and the heat-illness risk-index; and
the sensor unit being directly provided at the other end of the main body unit and fixed in place relative to the other end of the main body unit.

4. The moisture meter according to claim 1, further comprising:
a main body unit possessing one end and an other end opposite the one end;
a measurement-unit holding unit disposed at one end of the main body unit and configured to be positioned in the subject's armpit while sandwiched between an arm of the subject and a facing body part of the subject, the moisture measurement unit being located in the main body unit;
a display-unit holding unit disposed at the other end of the main body unit, with a display unit provided at the display-unit holding unit to display the amount of moisture of the subject measured by the moisture measurement unit and the heat-illness risk-index; and
the measurement-unit holding unit comprising a body-temperature measuring unit that measures body-temperature of the subject.

5. The moisture meter according to claim 4, wherein the display unit includes a body-temperature display portion which displays the body-temperature of the subject and the Wet-Bulb Globe temperature (WBGT) value.

6. A body moisture meter comprising:
a linear-shaped main body unit;
an insertion unit connected to the main body unit and extending in a forward direction from the main body unit, the insertion unit comprising a housing possessing a proximal end connected to the main body unit and an oppositely located distal end at which is located a distal surface of the housing;
a sensor unit which measures data indicating moisture inside a living body of a subject through contact with a body surface of the subject;
the sensor unit being movably mounted at the distal end of the housing of the insertion unit so that the sensor unit is movable in a movement direction approximately perpendicular to the distal surface and which outputs a signal instructing start of a measurement by the sensor unit upon detecting movement of the sensor unit in the movement direction; and
the distal surface of the insertion unit being configured such that an angle between a longitudinal extent of the main body unit and the movement direction of the sensor unit is 20° to 45°, and the insertion unit extends along the movement direction of the sensor unit.

7. The body moisture meter according to claim 6, wherein the housing possesses a lower surface which is curved toward the distal surface of the housing so that the lower surface is a concave surface when the main body unit is horizontally positioned.

8. The body moisture meter according to claim 7, wherein the insertion unit is connected to the main body unit at a boundary, and the insertion unit possesses a length such that a horizontal distance from the boundary to a center distal-most point of the sensor unit while the main body unit is horizontally positioned is 40 mm to 50 mm or 80 mm to 90 mm.

9. The body moisture meter according to claim 8, wherein the insertion unit is configured such that a cross-section area of the insertion unit becomes smaller toward the distal surface.

10. A body moisture meter comprising:
a sensor unit to be contacted with a body surface of an armpit of a subject and to output a signal identifying an amount of moisture inside a living body;
conversion means for converting the signal from the sensor unit to the amount of body moisture;
display means for displaying the amount of body moisture obtained by the conversion means; and
changing means for changing a display mode by the display means so as to call users' attention in a case in which the amount of body moisture obtained by the conversion means is lower than a first reference value, wherein
the first reference value is a value corresponding to a predetermined value between 25% to 40% in a case in which signals outputted when the sensor unit measures water and when it measures air are allotted 100% and 0% amounts of body moisture respectively in which the signal outputted by the sensor unit and the amount of body moisture are correlated by a linear relationship.

11. The body moisture meter according to claim 10, wherein the predetermined value is 35%.

12. The body moisture meter according to claim 10, wherein the changing means changes the display mode by the display means to still another mode in a case in which the amount of body moisture obtained from the conversion means is lower than a second reference value and the second reference value is a value smaller than the predetermined value.

13. The body moisture meter according to claim 12, wherein the second reference value is 25%.

14. The body moisture meter according to claim 11, wherein the conversion means sets a value corresponding to a predetermined value between 35% to 25% in a case in which signals outputted when the sensor unit measures water and when it measures air are allotted 100% and 0% amounts of water respectively in which the signal outputted by the sensor unit and the amount of water are correlated by a linear relationship.

15. A display control method of a body moisture meter that includes a sensor unit which outputs a signal indicating an amount of moisture inside a living body by being in contact with a body surface of an armpit of a subject, the method comprising:
converting the signal from the sensor unit to the amount of body moisture;
displaying on a display unit the amount of body moisture obtained from the converting; and
changing the display mode on the display unit to call a users' attention to a situation in which the amount of body water obtained during the converting is lower than a first reference value; and
wherein the first reference value is a value corresponding to a predetermined value between 25% to 40% in a case in which signals outputted when the sensor unit measures water and when it measures air are 100% and 0% amounts of body moisture respectively in which the signal outputted by the sensor unit and the amount of body moisture are correlated by a linear relation.
